(19) United States
(12) Patent Application Publication
(10) Pub. No.: US 2022/0299515 A1
(43) Pub. Date: Sep. 22, 2022

(54) Devices and Methods for Female Health Monitoring

(71) Applicant: Anna Villarreal, Brookline, MA (US)

(72) Inventor: Anna Villarreal, Brookline, MA (US)

(21) Appl. No.: 17/699,095

(22) Filed: Mar. 19, 2022

Related U.S. Application Data
(63) Continuation of application No. 63/163,801, filed on Mar. 20, 2021.

(51) Int. Cl. G01N 33/574; G01N 33/68; G01N 33/74; C12Q 1/6883; G01N 30/72; G01N 30/86; G16H 10/40; G16H 50/20

(52) U.S. Cl. CPC G01N 33/57488; G01N 33/6848; G01N 33/6893; G01N 33/743; C12Q 1/6883; G01N 30/7233; G01N 30/8624; G16H 10/40; G16H 50/20; C12Q 2600/156; C12Q 2600/158; C12Q 2600/118; G01N 2800/042; G01N 2030/027

(57) ABSTRACT

The present invention relates to improvements in female health monitoring and treatment wherein menstrual blood samples are analyzed for biomarkers of interests to monitor the health status and treatment of a female patient.

### FIELD OF THE INVENTION

This invention relates to, in part, methods and devices that are useful for the treatment, prevention, and/or diagnosis, of various diseases in females, including through repeated monitoring of various disease- or health-related biomarkers using menstrual fluid.

### BACKGROUND

Healthcare is estimated to account for nearly twenty percent of the U.S. GDP—an amount of almost three trillion US dollars. Considering the increased demand for healthcare, resulting from, for instance, increasing population sizes and life expectancies, the magnitude of this industry is expected to rise. With increased demand comes less access to health care practitioners either in the form of, for example, shorter visits or unavailability of appointments. Further, the increased reliance on biomarkers for monitoring patient health and prescribing pharmaceuticals, make accurate biomarker measurements critical for effective health care.

What is needed, are convenient and readily available methods, devices, and systems that provide accurate biomarker measurements, while not placing large demands on the health care system.

### SUMMARY OF THE INVENTION

Accordingly, the present invention provides methods and devices that enable females to engage in long-term health monitoring of important disease- or health-related biomarkers in are liable, cost effective, and non-invasive manner. Women are nine times more likely to develop an autoimmune disorder than men. Approximately 90 million women in the United States visit an OB/Gyn yearly. Worldwide, approximately 2 billion women are of menstruating age and approximately 85 million menstruating women are in the United States. Finally, approximately 75 percent of all clinical trials are not reported by sex. Menstrual blood is home to at least 385 unique proteins as well as many proteins and modifications that indicate diseases and disorders both inside and outside of the reproductive system.

In one aspect, the invention provides a method for evaluating one or more disease- or health-related biomarkers in a female subject, comprising: (a) obtaining a sample of the female subject's menstrual fluid; (b) measuring the presence, absence, or level of one or more disease- or health-related biomarkers in the sample by extracting or otherwise detecting the biomarkers in the sample; and (c) repeating with subsequent menstrual fluid. The trend and/or average levels of the biomarker(s) can be evaluated by the healthcare provider or patient to inform healthcare or lifestyle decisions, including in some embodiments the diagnosis of early stage disease or the state of chronic disease (e.g., controlled or uncontrolled) such as diabetes.

Diabetes is a global health issue that has been on the rise over the last several decades and is a major precursor to heart disease. In order to effectively address diabetes as a global health issue, and prevent further complications associated with the disease, we need to identify pre-diabetic patients; those who can make the necessary adjustments to their lifestyle to prevent diabetes, ensuring good health and reducing the burden on the health care system. “Most advice regarding healthy lifestyle, nutrition and exercise is good for both women and men, but we believe that some methods to prevent and treat heart attacks would be different in women than in men,” as one Johns Hopkin researcher stated who has been funded by the American Heart Association and AstraZeneca to study new ways to individualize the treatment and prevention of heart disease. As a preventative measure, the identification of prediabetic patients often does not occur, namely because they are otherwise healthy and lack access to adequate screening procedures. A disproportionate burden can fall on minority groups, especially mothers and women of color, and can therefore be at greater risk of being affected by diabetes and heart disease.

In diabetic patients there is a higher concentration of glucose in the blood with the consequent modification of hemoglobin, which is a long lived protein in circulation. This process is referred to as glycation, or the nonenzymatic addition of a sugar to a protein, in this case hemoglobin. The standard tests used for maintenance and diagnosis of diabetes monitor glycation of the HbA1c, or Beta-subunit of hemoglobin. This test monitors the amount of total glycation, not differentiating between the numbers of glycans added, thus is essentially considered to be based on one glycation event per hemoglobin. The HbA1C test, traditionally, is based on the following scale: 4-5.6% glycated is normal, 5.7-6.4% is high risk for developing diabetes, and greater than 6.5% is diagnosable as diabetic. Glucose reacts non-enzymatically with the N-terminus of the beta chain to form a stable ketoamine linkage. This takes place slowly and continuously throughout the 120-day life span of the red blood cell. The rate of glycation is increased in patients with diabetes mellitus. Based on the percentage of hemoglobin beta subunits with a glucose attached to the N-terminal valine of the beta subunit. For people without diabetes, the normal range for the hemoglobin A1c level is between 4% and 5.6%. Hemoglobin A1c levels between 5.7% and 6.4% indicate a higher chance of developing diabetes. Levels greater than 6.5% indicate a diagnosis of diabetes. In poorly managed or undiagnosed diabetes, levels may be as high as 15, 20, even 25%.

Our study describes a previously undocumented, multiple glycation event (from 1 to 4+) on the alpha subunits of hemoglobin. We observe this multiple glycation events on the alpha subunits of hemoglobin in multiple patient samples. The standard test for diabetes management is the HbA1c measurement, which measures the concentration of singly glycated beta subunits of hemoglobin.

In some embodiments, the present methods allow for long term health data that informs a healthcare provider in making healthcare decisions and/or providing improved health care and/or informs a female subject to make improved health/lifestyle decisions. In various embodiments, the method: unnecessary medical care visits, reduces or eliminates unnecessary diagnostic tests, reduce or eliminate unnecessary administration of therapeutic agents improve the selection of diagnostic tests and improve the selection of therapeutic agents.

Further, in various embodiments, the present methods provide baseline biomarker levels for the patient, as well as long term and short term trends in biomarker levels. Such baseline information or trends allow for more accurate and interpretable diagnostic and/or prognostic tests including, for example, when the baseline or trend health information is used to compare to a biomarker measurement at a single point of time (including, by way of non-limiting example, at a point of care, e.g., upon visit to a healthcare profession presenting symptoms of a disease or disorder).

In one form of the present invention, a laboratory developed test (LDT) is used to provide health information to a patient. A laboratory developed test is a type of in vitro diagnostic test that is designed, manufactured and used within a single laboratory. LDTs can be used to measure or detect a wide variety of analytes (substances such as proteins, chemical compounds like glucose or cholesterol, or DNA), in a sample taken from a human body. Some LDTs are relatively simple tests that measure single analytes, such as a test that measures the level of sodium. Other LDTs are complex and may measure or detect one or more analytes. For example, some tests can detect many DNA variations from a single blood sample, which can be used to help diagnose a genetic disease. Various levels of chemicals can be measured to help diagnose a patient's state of health, such as levels of cholesterol or sodium. While the uses of an LDT are often the same as the uses of FDA-cleared or approved in vitro diagnostic tests, some labs may choose to offer their own test. For example, a hospital lab may run its own vitamin D assay, even though there is an FDA-cleared test for vitamin D currently on the market. LDT's are important to the continued development of personalized medicine, so it is important that in vitro diagnostics are accurate so that patients and health care providers do not seek unnecessary treatments, delay needed treatments, or become exposed to inappropriate therapies.

In various embodiments, the present invention provides a device, such as a disposable cartridge for collecting biomarker information, and optionally inserted or insertable into a wireless enabled device, such as a personal communication device. The invention further provides methods of using cartridges and systems. Accordingly, in various embodiments, the wireless enabled device may link to the cloud and allow secure access to the biomarker information by the patient and one or several healthcare providers and/or diagnostic service providers, or other parties (including health and/or life insurance providers). In some embodiments, the present invention provides a database of the female subject's biomarker information, which may be locally or remotely stored, including cloud-based. Also, in some embodiments, the system comprising the female subject's biomarker information further comprises user interfaces (e.g. graphical user interfaces) that allow controlled and/or secure access to the information. Such interfaces may be accessed via an application on a personal communication device.

In various embodiments, the methods provided herein comprise measurement via extraction or ode methods of detecting biomarkers of various disease- or health-related biomarkers that are used to direct healthcare decisions and/or personal health decisions. In particular, the present invention provides for biomarker measurements for which long term data is desirable. For example, biomarker measurements that are hindered by inconsistency when measured in a single point in time (e.g. point of care) scenario are provided. Further, disease- or health-related biomarkers that are surrogates for slowly developing and/or relatively symptom-free, and/or chronic diseases are provided. In one form of the present invention, a detailed report is provided showing the levels and trends for the selected biomarkers. For the convenience of the patient, the results may be shown using various colors or graphs to indicate the desired biomarker levels and areas of concern for the patient. This report may then be used by the patient to seek further testing or treatment from a medical professional.

### BRIEF DESCRIPTION OF THE DRAWINGS

FIGS. 1A and 1B are illustrative of the glycated alpha subunit Hemoglobin from menstrual blood and fingerpricks, respectively;

FIG. 2 is a baseline chart of HbA1c analyzed for glycation;

FIG. 3 is a chart of menstrual blood at baseline for multiple patients;

FIG. 4 is a chart of menstrual blood and fingerprick blood samples analyzed for glycation at 3 months following baseline;

FIG. 5 is a chart of menstrual blood and fingerprick blood samples analyzed for glycation at 6 months following baseline;

FIG. 6 is a chart of menstrual blood and fingerprick blood samples analyzed for glycation at 12 months following baseline;

FIG. 7 is a chart of menstrual blood and fingerprick blood samples analyzed for glycation at 24 months following baseline;

FIG. 8 is a chart of menstrual blood and fingerprick blood samples analyzed for glycation at 36 months following baseline;

FIG. 9 is a chart of menstrual blood and fingerprick blood samples analyzed for glycation at 48 months following baseline;

### DETAILED DESCRIPTION OF THE INVENTION

The present invention is based, in part, on the discovery that improved healthcare or personal health maintenance for a variety of conditions can be achieved by periodic evaluation of disease- or health-related biomarkers in menstrual fluid.

In one aspect, the invention provides a method for evaluating one or more disease- or health-related biomarkers in a female subject, comprising obtaining a sample of the female subject's menstrual fluid: measuring the presence, absence or level of one or more disease- or health-related biomarkers in the sample, and repeating the testing with subsequent menstrual fluid samples. The trend and/or average levels of the extracted, measured and/or detected biomarker(s) can be evaluated by the healthcare provider or patient to inform healthcare or lifestyle decisions (or in some embodiments, decisions of an insurance provider), including in some embodiments the diagnosis of early stage disease or the state of chronic disease (e.g., controlled or uncontrolled).

As described herein, the detection of the glycation of hemoglobin as an evaluation of the predisposition of a female patient to diabetes is provided. Furthermore, subsequent sampling may be used to detect the progression towards clinical diabetes. The menstrual fluid of a patient is analyzed to determine the concentration and formulation of the glycated hemoglobin. Similarly, the menstrual fluid may be analyzed for the presence and level of other biomarkers of interest and may be correlated to detect or diagnose other disease states.

In various embodiments, the present methods provide for repeated sampling of a female subject's menstrual fluid to allow for an accumulation of data over a period of weeks, months or years. Such data is used to form a more complete subject's health history than standard point of care testing. As described herein, such data allows for an improvement in personal healthcare and/or health decisions.

Also, in some embodiments, the present invention provides for a non-invasive method od monitoring one's health. For example, in various embodiments, the collection of menstrual fluid provides biomarker information without the need for blood draws, biopsies, etc. In some embodiments, the present methods allow for long term health monitoring without various deleterious side effects of standard monitoring including, by way of illustration, excessive bleeding, fainting, lightheadedness, hematoma, infection, pricking or stinging sensations, bruising, pain, throbbing, etc. In some embodiments, the non-invasive nature of the sample collection improves patient compliance and allows for a more complete set of data.

In various embodiments, the evaluation informs a healthcare provider to provide improved health care and/or informs the female subject to make improved health decisions. For example, subtle alterations in one or more disease- or health-related biomarkers over time, away from a normal level, may provide an earlier indication of a disease or disorder than a test at a single point of time (including, by way of non-limiting example, at a point of care) test and before symptoms arise. Further, the repeated evaluations of the present methods allow for early detection of a disease or disorder as the evaluation is not driven by a symptom or sign on the subject part. For instance, the repeated evaluation of menstrual fluid allows for increased healthcare vigilance and largely eliminates the need for reactive medical interventions. In some embodiments, the subject has a chronic disease such as diabetes, congestive heart failure, or multiple sclerosis, and the state (e.g., controlled or uncontrolled) is monitored over time. In some embodiments, the patient has a history, or family history, of cancer, and the method allows recurrence of disease to be closely monitored.

In some embodiments, the evaluation comprises any one of diagnosis, prognosis, and response to treatment. Diagnosis refers to the process of attempting to determine or identify a possible disease or disorder. Prognosis refers to the predicting of a likely outcome of a disease or disorder. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment (e.g. response to a therapeutic agent). Responses to treatment can be, by way of non-limiting example, pathological complete response, survival, improvement and/or remission.

In various embodiments, the present invention pertains to the generation of along term health history record that informs care. Accordingly, in various embodiments, the menstrual sample is obtained periodically. In some embodiments, the menstrual sample is obtained on a regular basis. For instance, sampling may occur about once every month, or about once every other month, or about once every 3 months, or about once every 6 months, or about once every 9 months, or about once every year. In some embodiments, about 1 to about 12, or about 2 to about 10, or about 3 to about 8 samples are evaluated per year.

Furthermore, in some embodiments the present methods are repeated long term to generate a large dataset. For example, in some embodiments, the evaluation is repeated for about 3 months, or about 6 months, or about 9 months, or about 1 year, or about 2 years, or about 3 years, or about 4 years, or about 5 years, or about 6 years, or about 7 years, or about 8 years, or about 9 years, or about 10 years, or about 20 years, or about 30 years, or about 40 years, or about 50 years.

In various embodiments, the female subject's biomarker information provides baseline health information, as well as long term and short term trends in biomarker levels. In various embodiments, the baseline or trend health information is used to compare to a biomarker measurement at a single point in time (e.g. at the point of care). For example, in various point of care settings, a diagnostic test may not be informative because the single data point being taken may not be reflective of the female subject's condition (e.g. in tests that are prone to data obfuscation by various lifestyle effects, by way of illustration CEA (carcino-embryonic antigen) readings may be skewed by smoking). The present repeated evaluation establishes a baseline or trend value to which comparison can be made. In one embodiment, the female subject is symptomatic for a disease or disorder and a standard single point of time (including, by way of non-limiting example, at a point of care) diagnostic test is taken. Such single point of time test may be a blood test that need not be menstrual fluid. This data is compared to the long term data on the same biological parameter to establish if there is a meaningful change that indicates a disease or disorder. Such information dictates whether further testing is required or if certain treatments should be administered. Accordingly, in some embodiments, the present methods prevent or mitigate incorrect or missed diagnosis. In various embodiments, the present methods allow for one or more of reducing or eliminating unnecessary medical care visits, reducing or eliminating unnecessary diagnostic tests, reducing or eliminating unnecessary administration of therapeutic agents, improving the selection of appropriate diagnostic tests, and improving the selection of appropriate therapeutic agents.

In various embodiments, the present invention relates to various disease- or health-related biomarkers that are available in menstrual fluid. For example, the present invention provides for disease- or health-related biomarkers for which long term data is desirable. Further, in some embodiments, the disease- or health-related biomarkers of the present invention are those which are hindered by inconsistency when measured in a single point of time (including, byway of non-limiting example, at a point of care) scenario. Further still in some embodiments, the present disease- or health-related biomarkers include those which are surrogates for slowly developing and/or relatively symptom-free and/or chronic diseases.

An example of samples preparation and analysis includes the steps set forth below.

### I. MS Analysis of Intact Proteins in Menstrual Fluid

### Overview

This procedure was used to identify the intact masses of hemoglobin and other proteins in menstrual fluid samples. Using this method, intact hemoglobin species with various post-translational modifications can be observed, including multiple glycations on both the alpha and beta subunits.

Extraction of Menstrual Fluid from Collection Paper

Menstrual fluid-soaked filter paper portion (a spiral 6 cm in diameter) was extracted with 200 ul of 100 mM ammonium bicarbonate in a 1.5 ml Eppendorf tube. It was shaken at room temperature, 1000 RPM for 45 min. A 30 uL aliquot of the eluate was directly analyzed via intact mass spectrometry.

### UPLC-MS Method

Samples were separated on a C-4 reverse phase column over the course of 100 minutes. They were then injected into the ESI-Q-Tof Mass Spectrometer using standard parameters for MS analysis. Total ion chromatograms were generated from the runs and used to identify specific protein masses. The mass spec data collected under a specific peak, at a specific retention time, was analyzed by converting the m/z to molecular mass using the Maximum Entropy Deconvolution algorithm. Post-translational modifications were observed manually by identifying a mass shift from the unmodified protein peak; tentative protein assignments were determined by their mature protein mass, and verified via an MS/MS analysis of trypsin digested menstrual fluid (See next section).

### II. In-Gel Trypsin Digestion and MS/MS Analysis of Menstrual Fluid Proteins

### Overview

This procedure was employed to identity particular proteins found in the menstrual fluid sample. Using this method, over 130 proteins species were detected, including hemoglobin subunits alpha and beta.

Extraction of Menstrual Fluid from Collection Paper

Menstrual fluid-soaked filter paper portion (a spiral 6 cm in diameter) was extracted with 200 ul of 100 mM ammonium bicarbonate in a 1.5 ml Eppendorf tube. It was shaken at room temperature, 1000 RPM for 45 min.

### In-Gel Digestion of Menstrual Blood Proteins

### Gel Electrophoresis and Gel Excision

Samples were vortexed at 3000 rpm for 30 seconds and then centrifuged for 2 minutes. A 10 uL aliquote was mixed with 10 uL of 2× Laemmli loading dye and heated at 99 C for 5 minutes. The samples were separated on a 4-20% TGS Precise gel @125V for 1.5 hours. The entire gel was rinsed 3 times in water and then covered with gel staining dye and shaken for 30 minutes. The gel was then rinsed again with water and destained in water for 30 minutes. Each lane was the excised and divided in to 10 equally sized pieces. Each of those pieces was then chopped in smaller pieces and added a 1.5 mL Eppendorf tube. If these pieces contained large amounts of visible dye, they were further destained by alternating the addition of acetonitrile and then water to remove this additional stain. Finally, the pieces were shrunken with acetonitrile to prepare them for the reduction and alkylation steps.

### Reduction and Alkylation

To each tube, 50 uL of 10 mM freshly prepared dithiothreitol was added and allowed to incubate for 30 minutes at 56 C. The supernatant was removed and the gel was shrunk with 200 uL of acetonitrile while shaking. 50 uL of 55 mM freshly prepared iodoacetamine was then added and allowed to incubate at room temperature for 60 minutes in the dark. The gel was the washed once with 200 uL of 100 mM ammonium bicarbonate for 15 minutes, with shaking. The supernatant was then removed and the gel shrunk with 200 uL acetonitrile.

### Trypsin Digest

20 ng of lyophilized trypsin was prepared by adding 20 uL of 50 mM acetic acid and mixing by pipetting. 10 uL of this stock solution was added to 790 uL of 50 mM ammonium bicarbonate with 5 mM CaCl₂), to make 12.5 ng/uL trypsin. 50 uL of this solution was added to each tube with shrunken gel pieces and incubated at 4 C for 35 minutes. The supernatant was discarded and 50 uL of the buffer alone was added to cover the gel pieces. These were then incubated overnight at 37 C. The supernatant was collected and 100 uL of 50 mM ammonium bicarbonate was added to the sample and shaken for 15 minutes. This was then centrifuged for 5 minutes and the supernatant collected. 150 uL of acetonitrile as added and incubated for 15 minutes and the supernatant was collected. To stop the digest, 30 uL of 5% formic acid was added to the gel and shaken for 5 minutes. 100 uL of acetonitrile was added to the gel and the entire supernatant was collected and added to the entirety of the former extractions. The extraction was then dried down to 30 uL at 35 C.

### UPLC-MS Method

Samples were separated on a C-18 reverse phase column over the course of 90 minutes. They were then injected into the ESI-Q-Tof Mass Spectrometer using standard parameters for MS analysis. The mass spec data collected in an entire sample run was exported to a mascot generic file (.mgf) and searched against the SwissProt database through MASCOT software. Proteins were identified by sequence; a spectral count of 5 is considered significant.

### Data Interpretation

All MS/MS peptide fragmentation data was interpreted using MASCOT software. Relevant parameters for identifying proteins from which the peptide were derived include specifying a search from the SwissProt protein database. Up to 3 missed cleavages were allowed.

### III. In-Solution Digestion and MS/MS Analysis of Glu-C and Trypsin Digested Hemoglobin in Menstrual Fluid

### Overview

This procedure was employed to identity the location of particular glycation events on hemoglobin subunit alpha, found in the menstrual fluid sample. Using this method, multiple specific locations of glycation were identified.

### In-Solution Digestion of Menstrual Blood Proteins

Extraction of Menstrual Fluid from Collection Paper

Menstrual fluid-soaked filter paper portion (a spiral 6 cm in diameter) was extracted with 200 ul of 100 mM ammonium bicarbonate in a 1.5 ml Eppendorf tube. It was shaken at room temperature, 1000 RPM for 45 min.

### Trypsin Digest

20 ng of lyophilized trypsin was prepared by adding 20 uL of buffer solution and mixing by pipetting. This solution was added to 20 uL of sample and incubated for 2 hours at 37 C. This sample was then subjected to MS/MS analysis.

### Glu-C Digest

10 ng of lyophilized Glu-C was prepared by adding 10 uL of buffer solution and mixing by pipetting. This solution was added to 10 uL of sample and incubated for 2 hours at 37 C. This samples was then subjected to MS/MS analysis.

### UPLC-MS Method

Samples were separated on a C-18 reverse phase column over the course of 90 minutes. They were then injected into the ESI-Q-Tof Mass Spectrometer using standard parameters for MS analysis. The mass spec data collected in an entire sample run was analyzed via the UNIFI software package.

### Data Interpretation

All MS/MS peptide fragmentation data was interpreted using UNIFI software. Identification of peptide fragments containing K-linked glycation events were identified and cross matched between the alternative digest methods.

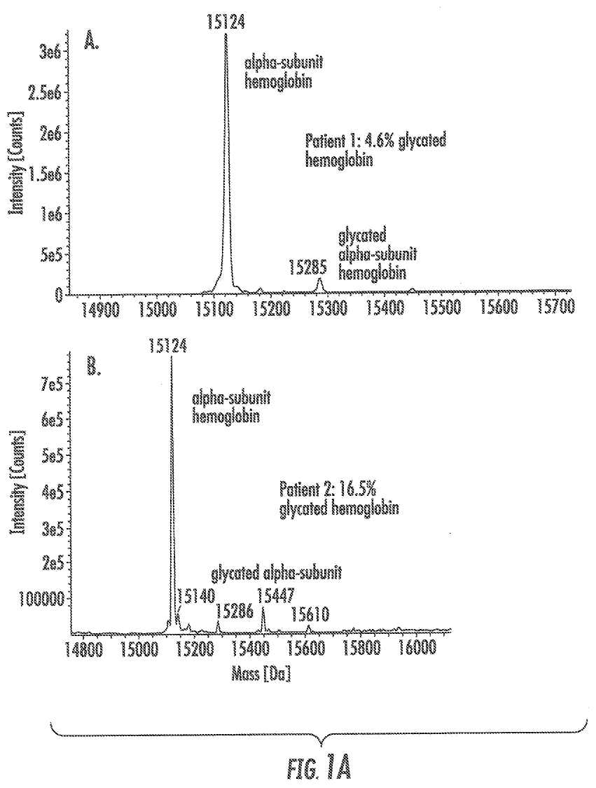

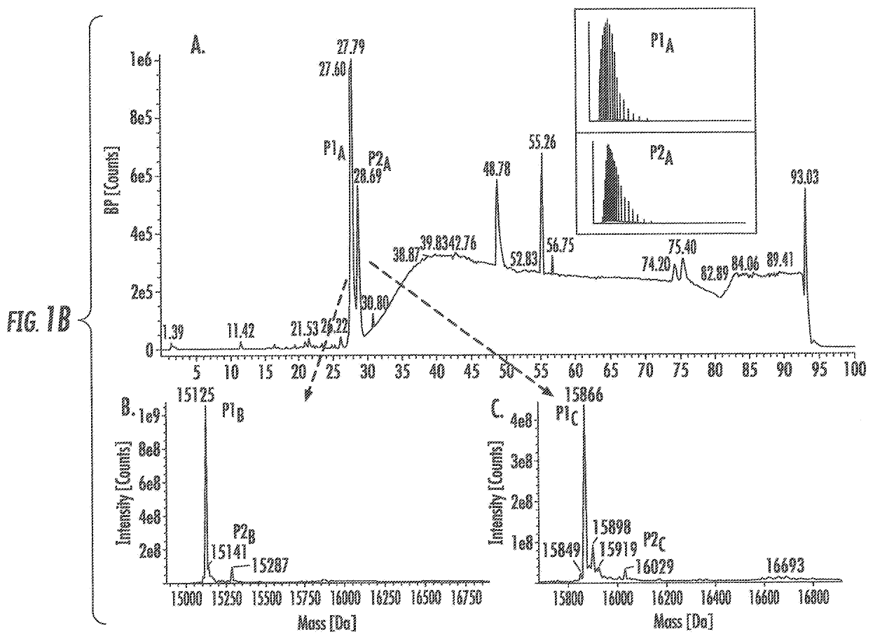

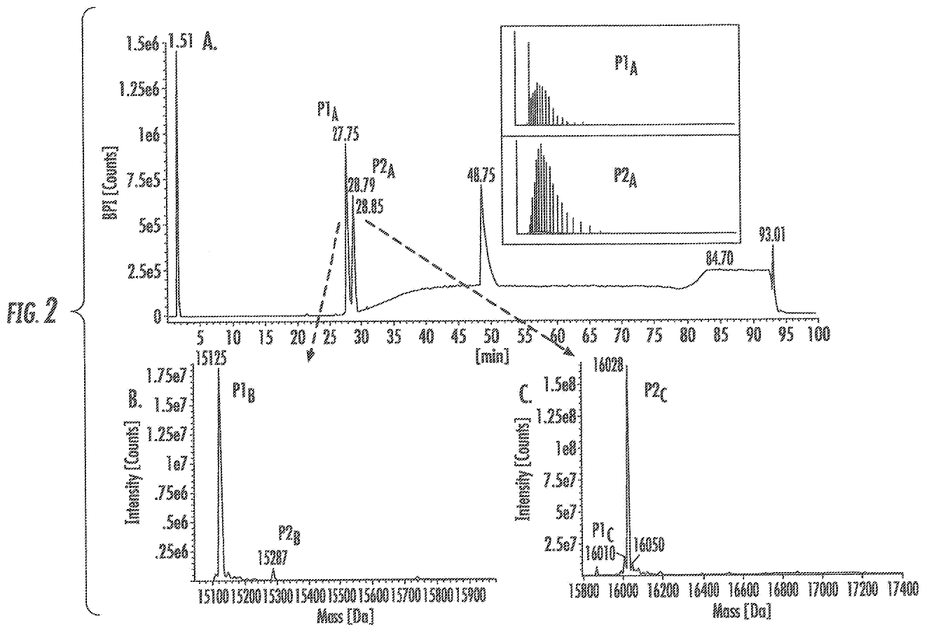

Our study describes a multiple glycation event (from 1 to 4+) on the alpha subunits of hemoglobin as shown in FIGS. 1A and 1B. FIGS. 1A and 1B are illustrative of how menstrual blood based proteomics differ from proteins found in peripheral blood-based and pure lab derived proteins. We observe this multiple glycation events on the alpha subunits of hemoglobin in multiple patient samples as shown in FIG. 1A for a selected patient. FIG. 1B is an intact mass spectrum showing hemoglobin subunit alpha and beta as P1 and P2. The deconvoluted drawings show an unmodified beta subunit and a singly glycated hemoglobin. The standard test for diabetes management is the HbA1c measurement, which measures the concentration of singly glycated beta subunits of hemoglobin. FIG. 2 shows a total ion chromatogram with intact mass spectrum showing the hemoglobin subunit alpha and beta.

### Method

We obtained an authenticated 96% natural HbA1c sample (abcam ab98306) which under our LC-MS conditions we observe as separate alpha and beta subunits. The hemoglobin A1c is a native protein and prepared from packed red blood cells; the protein arrives intact. The tetramer molecular weight is ˜64 KDa consisting of 4 subunits of ˜16 KDa each. Our analysis shows this standard exhibits a level of 5.88% glycated hemoglobin subunit alpha. It shows no signs of multiple glycation sites. It also exhibits 95.4% glycated subunit beta, as authenticated.

### Results and Discussion

Menstrual Blood Proteome Analysis and its unique signatures for possible early detection of prediabetes or progression towards a diagnosis of diabetes.

### Intuitive Analysis for Magnitude of Glycation of Hemoglobin

For Sample 1, When RT is 22.5 to 27.5 and the BPI count of the sample is around 100000 the hemoglobin Subunit Alpha is detected. When a sample is calculated for glycated hemoglobin unit and intensity of the molecule is around 326 and mass is 15124, it is said to be normal hemoglobin Subunit Alpha, apart from that, the mass 1 of glucose is detected when DA that is weight of molecule is 15285 and intensity of that mass is less than 5e5, second molecule of glucose is detected when mass weight is between 15400 to 15500 and intensity is very low and third glucose is detected when weight of mass is around 15601, when no intensity or little intensity is impacted on the molecule. All total 4.6% of glycated hemoglobin alpha unit is detected which means the person is not a diabetic person.

For sample 2, the RT Time is 27.42 and BPI count is around 40000, when hemoglobin subunit alpha is detected. When Intensity is greater than 7e5 and weight of molecule is 15124, normal hemoglobin subunit alpha is found to be present. The first molecule of glucose is present where intensity lower than 10000 and weight around 15286, second glucose molecule again with low intensity and when weight is 15447, and third one when weight is 15610. Total 16.5% glycose is detected making that person diabetic. When we compare both the samples, we can see that the person with no diabetes molecules when passed for testing take less retention time, but BPI count is higher for that molecule, whereas for the person who is having diabetes have greater RT time and less BPI count.

We begin to monitor a patient for prediabetes when they have more than three alpha glycations and 0 beta and/or 2+ alpha plus beta glycations. Once they reach a sustained measure of 3-4 alpha glycations, we recommend diabetes testing as it is an easy and straightforward test. To begin to discern diabetes from other diseases that could be causing inflammation (such as cancer) we compare fingerprick hemoglobin beta data as well as other health profile factors. For instance, out of 500 patients, 22 never exercise and their fingerprick glycation is relatively higher than other individuals. When exercise frequency is combined with BMI and fingerprick glycation and menstrual blood alpha glycation we find positive association to diabetes.

Individuals with BMI between 20 to 30 mostly have menstrual blood alpha glycation event (MBAGE) values around 1, followed by MBAGE values of 3. Also individuals with hypertension and glycation end products have median BMIs ranging between 26 to 28 and only a smaller number of Individuals are obese in this condition. Thus, we monitor over time the individuals with 3 glycations for prediabetes as well as those with hypertension and glycation end products. We recommend obese individuals with both glycation and hypertension undergo medical testing for diabetes. We attribute those with 1 glycation as low-risk for the disease unless it is correlated with BMI or other changes.

We also attribute age to an increase in menstrual blood alpha glycation levels which plays into age related disease(s). Individuals with more incidence of glycation events and 75th percentile of them are above 35 and close to 45 in age. Individuals with MBAGES 0 are younger than individuals with MBAGE 1, MBAGES 2, MBAGE3 and MBAGE4. Similarly, when looking at menstrual blood there is no evidence shown that taking birth control affects the MB glycation events increase or decrease. Correlation the between MB event and birth control appears to be independent. Additionally, these is a positive correlation to a combination of high blood pressure and depression increasing the hemoglobin beta in a fingerprick sample. This correlation was not present in the analysis of menstrual blood. The present study shows a increase in MB glycation and allows for lifestyle changes or medical methods allow for an noninvasive manner to monitor this. For example, if the periodic evaluations show MB glycation levels rising over time, medical intervention of lifestyle change may be ordered to mitigate detrimental effects (e.g. increased likelihood or onset of one or more of eye disease, heart disease, kidney disease, nerve damage, and stroke).

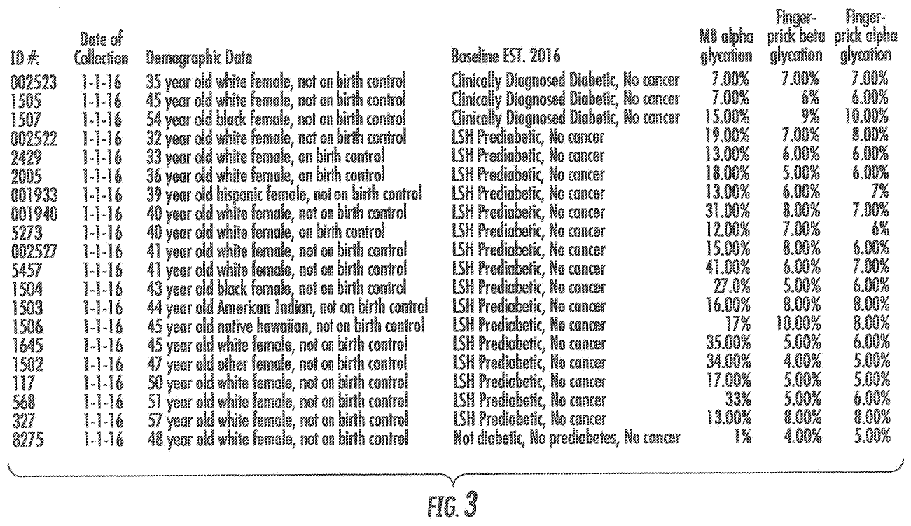

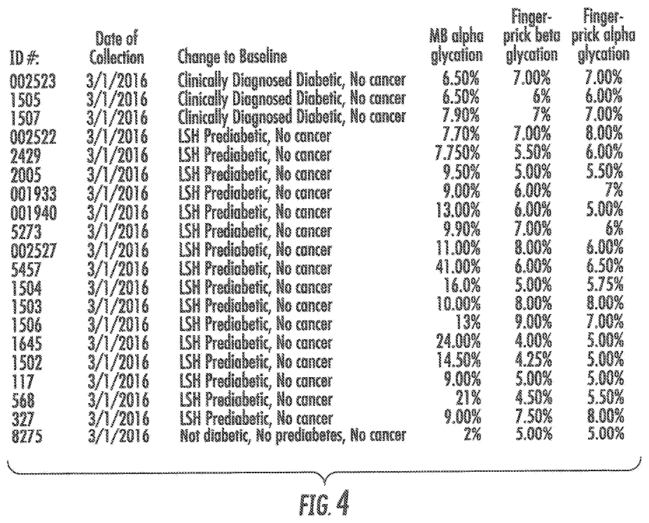

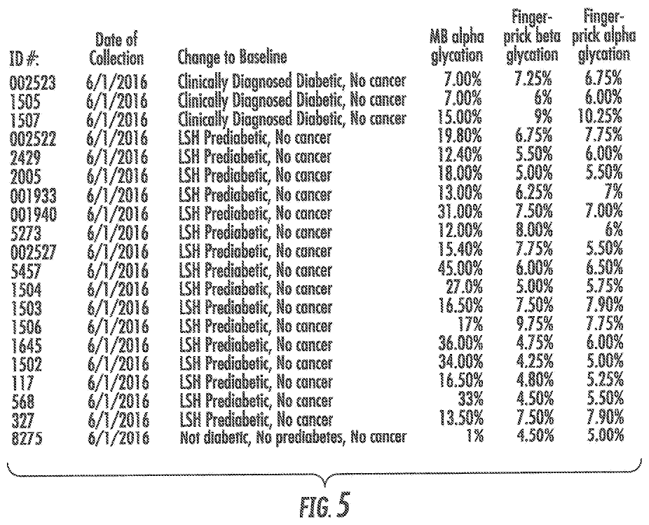

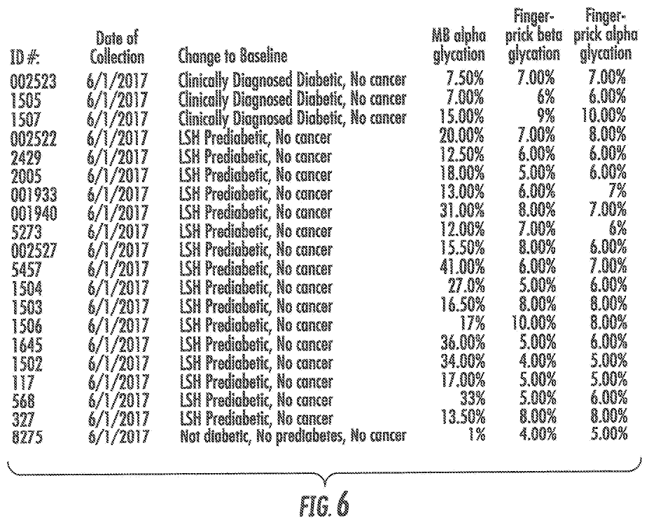

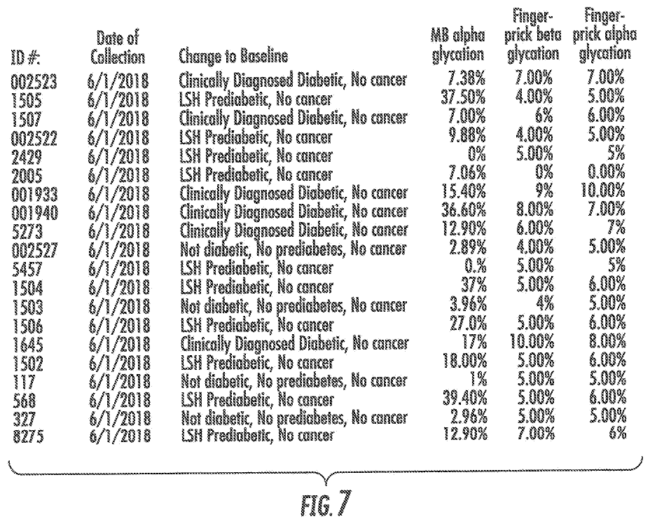

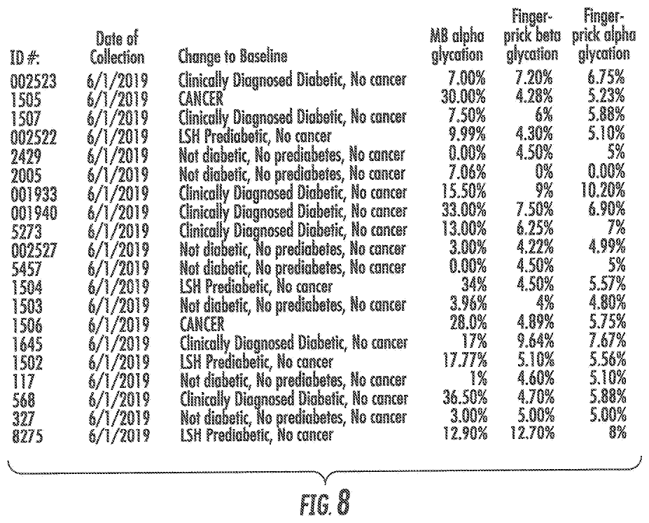

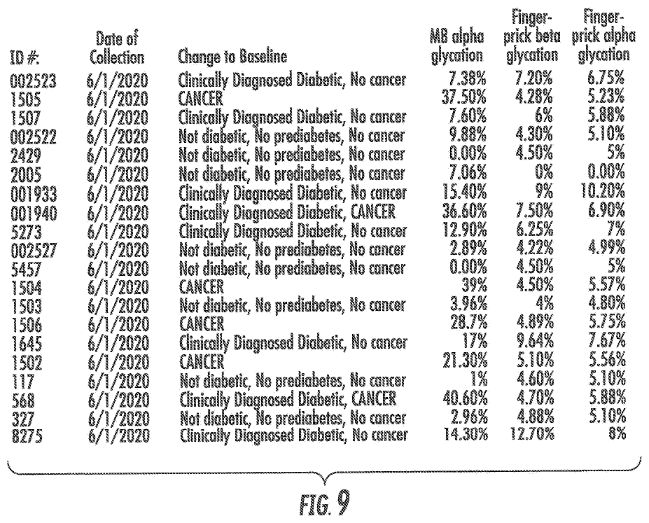

According to the study, a person with no diabetes has total 4.6% of glucose present which is normal and while a person with diabetes has 16.5% glycose present to support the diagnosis of diabetes. While reviewing the “Alpha subunit Hb” data, we note that the older a patient is, the higher number of modifications in their hemoglobin. We found that modifications to hemoglobin subunit alpha increase in women starting at age 35 and older. The charts set forth in FIGS. 3-9 are illustrative of the progression of a disease state using periodic menstrual blood samples.

In some embodiments, a panel of disease- or health-related biomarkers is employed. For example, in some embodiments, one or more of the disease- or health-related biomarkers described herein may be evaluated repeatedly. For example, at least 1, or 2, or 3, or 4, or 5, or 6, or 7, or 8, or 9, or 10, or 15, or 20, or 30, or 40, or 50 disease- or health-related biomarkers may be evaluated in a panel.

In various embodiments, the present methods include various disease- or health-related biomarkers including blood cells, vitamins and minerals, blood lipids, steroids, nitrogen markers, tumor antigens, miRNAs, and antibodies.

In various embodiments, the present methods include screening of various blood panels used in routine screening. For example, the present methods may relate to the complete blood count (CBC) panel and/or a blood chemistry panel and/or a blood gas panel. For example, such monitoring may assist nearly diagnosis of various conditions, such as anemia, infection, inflammation, bleeding disorders or cancers (e.g. leukemias).

In some embodiments, the present methods relate to repeated blood cell monitoring. For example, the following disease- or health-related biomarkers (including measuring the number, variety, percentage, concentration, and quality of blood cells) may be repeatedly monitored: red blood cells count, hemoglobin, hematocrit, red blood cell indices, MCV (mean corpuscular volume), MCH (mean corpuscular hemoglobin), MCHC (mean corpuscular hemoglobin concentration) are particularly suited for repeated evaluation. For example, monocyte, eosinophil, and basophil counts are rarely informative when taken as a single test; however, a trend of low counts of these cell types can indicate one or more cancers and bone marrow deficiencies, for example.

In various embodiments, the disease- or health-related biomarkers of the present invention are one or more of those on a standard blood chemistry panel. Accordingly, in some embodiments, the biomarker tested, and the condition evaluated, is one or more of those described herein. For example, the present methods may include one or more of the following: glucose, uric acid, BUN (blood urea nitrogen) (e.g. for liver and kidney function), creatinine (e.g. for kidney function), BUN/creatinine Ratio (e.g. for impaired renal function), estimated glomerular filtration rate (eGFR), sodium, potassium, chloride, calcium, phosphorus, total protein, albumin, globulin, albumin/globulin ratio, bilirubin (e.g. for kidney and liver function), alkaline phosphatase (e.g. for liver and bone diseases), LOH (lactate dehydrogenase), AST (SGOT)(e.g. for liver function), ALT (SGPT) (e.g. for liver function), iron (serum), and lipid profile (e.g. for the risk for developing atherosclerosis (arterial plaque) and coronary heart disease (including one or more of: total cholesterol, triglycerides, HOL cholesterol, LOL cholesterol, and total cholesterol/HOL ratio)).

In some embodiments, the estrogen levels of a patient are monitored using one or more menstrual samples. The understanding of the action of estrogen in the human has been confined to the regulation of ovulation and reproductive functions in the female. However, new evidence of estrogen's role in disease is emerging as scientific exploration into autoimmune disease, neurodegenerative disease, heart disease and diseases of the reproductive system continues to grow. As a result of the intrinsic differences in hormones between women and men, the role of hormones have been linked to increase the incidence of various diseases in women; leading to the role of cyclic estrogen signaling and the subsequent spike in estrogen production each month during menstruation, and its effects over time.

In premenopausal women the ovaries represent by far the most important and largest source of circulating estrogens. New research points to estrogen and estrogen signaling role that exceeds the classical endocrine regulatory role—beyond reproductive functions—including effects on the cardiovascular system, skeletal homeostasis, and the central nervous system. The value of using menstrual blood is considered important due in part to its unique spatial relationship to not just the reproductive system but also to the main site of estrogen production, the ovaries.

Sex-bias is evident in autoimmune diseases especially systemic lupus erythematosus (SLE) but the reason for this bias is yet to be understood. Strong evidence for the role of hormones in the pathogenesis of SLE is highlighted by the age at which the incidence rates peak. “Adult premenopausal female to male ratio of SLE is 9:1 and is closer to 2:1 during childhood or post menopause.” Research continues to show that estrogen causes a heightened immunoactivated state which possibly contribute to the inflammatory responses leading to autoimmune development. Females of child-bearing age are more resistant to infectious disease and have an increased risk of systemic lupus erythematosus (SLE). We hypothesized that estrogen-induced gene expression could establish an immunoactivated state which would render enhanced defense against infection but may be deleterious in autoimmune development. Using peripheral blood mononuclear cells (PBMCs), we demonstrate enhanced responses with immunogen stimulation in the presence of 17β-estradiol (E2) and gene array analyses reveal toll-like receptor 8 (TLR8) as an E2-responsive candidate gene. TLR8 expression levels are up-regulated in SLE and PBMCs stimulated with TLR8 agonist display a female sex-biased, E2-sensitive response. Moreover, we identify a putative ERα-binding region near the TLR8 locus and blocking ERα expression significantly decreases E2-mediated TLR8 induction. Our findings characterize TLR8 as a novel estrogen target gene that can lower the inflammatory threshold and implicate an IFNα-independent inflammatory mechanism that could contribute to higher SLE incidence in women.

In other research, researchers have found that estrogen signaling accelerates the progression of different estrogen insensitive tumor models. This progression was found to be a result of deregulated myelopoiesis—the production of bones and all the cells that arise from it (ie. All blood cells). Deregulation of myelopoiesis was found to be caused by the increase in mobilization of myeloid-derived suppressor cells (MDSCs; immune cells from bone marrow stem cells) and increased immunosuppressive activity. On a molecular level, researchers saw that estrogen receptor alpha activated the STAT3 pathway in human and mouse bone marrow myeloid precursors by enhancing the JAK2 and SRC activity, highlighting estrogens role in the mechanisms leading to pathological myelopoiesis in cancer.

Proteomic research has found STAT3 and JAK2 in menstrual blood and are of interest to our research whether with the identification of STAT3/JAK2 proteins in menstrual blood, changes in concentration of STAT3/JAK2 cascade proteins, respective genes responsible for STAT3/JAK2 proteins, protein networks, and correlations between these proteins and disease state.

Ovarian cancer is the deadliest gynecologic malignancy in women worldwide for reasons that include late-stage diagnosis at advanced tumor stage and rapid resistance to chemotherapy. Data is starting to show that the onset and the biology of ovarian cancer is directly correlated to lifetime estrogen-exposure. Additional research highlights the similarities between ovarian cancer cells estrogen regulated pathways to other cancers such as endometrial cancer and breast cancer. An additional obstacle is presented when taking into consideration the number of various mutations, origins, metastatic behaviors and responses (or lack thereof) to chemotherapies—requiring a strong understanding of all subtypes of ovarian cancer.

To better understand the effects of estrogens two hypothesis are presented—1) the “incessant ovulation hypothesis” from 1971 and 2) the most recent “incessant menstruation hypothesis”. The incessant ovarian hypothesis suggests that ovarian cancer is the result of “repetitive wounding during ovulation and the subsequent activation of repair mechanisms”, and as a result of increased number of mutations that have accumulated in epithelial cells. The incessant menstruation hypothesis which suggests that high-grade serous ovarian cancer is derived from cells in the fallopian tubes, floating in bloody peritoneal fluid. As a result of retrograde menstruation taking place every month, they are repeatedly exposed to iron-induced oxidative stress as a result of the hemolysis of blood cells by pelvic macrophages found at the distal site of the fallopian tubes. Taking this theory into consideration, the cyclical nature of menstruation in combination with the effects of estrogen and estrogen signaling together present a unique opportunity for the biological development of a tumor-network, thus allowing for cancer and other diseases to develop at greater rates in women than in men.

The tumor promoting effects of estrogen are broken down into 2 categories:

1) Receptor-dependent mechanisms—describing the effects of binding of estrogen to ER alpha and its subsequent transcriptional effects.
2) Receptor independent mechanisms—describing the effect of free-radicals generate by the activation of estrogen and the subsequence accumulation of mutations in the fallopian tube and the ovaries. With this being said, analysis of menstrual blood should include the induction of ERK, PI3K and EGFR cascade signaling as a result of G-protein coupled estrogen receptor families and estrogen metabolism (each month during menses) that leads to the formation of free radicals, and the accumulation of mutations in distal portions of the fallopian tubes.

Metastatic breast cancer is a life-threatening stage of cancer and is the leading cause of death in advanced breast cancer patients. Estrogen signaling and the estrogen receptor (ER) are implicated in breast cancer progression, and the majority of the human breast cancers start out as estrogen dependent. Accumulating evidence suggests that ER signaling is complex, involving coregulatory proteins and extranuclear actions. ER-coregulatory proteins are tightly regulated under normal conditions with miss expression primarily reported in cancer.

The American Heart association states that a decline in the natural hormone estrogen may play a role in heart disease among post-menopausal women. Cardiovascular disease is the number one cause of death among women, accounting for nearly 50% of female deaths. Statistics show that women on average develop cardiovascular disease 10 to 15 years later in life than men, and that the risk may increase after menopause. This observation has led to much speculation as to what physiological change(s) associated with menopause is responsible for the higher risk of atherosclerosis. Estrogen, with its potential as a cardioprotective agent and as an immunomodulator of the inflammatory response in atherosclerosis, has received significant attention.

In another embodiment, our intention was to identify a target protein, one that possibly plays a role as a cancer biomarker. We have identified glycodelin as a cancer biomarker in menstrual fluid after reviewing tryptic digests and MS/MS analysis of peptides from the first 7 samples. In one of the menstrual blood samples we tested, we detect glycodelin. Of the proteins we have consistently detected, glycodelin is a good target to investigate further for several reasons:

1. We observe it in 2 of our tryptic digest analyses.
2. In excised band 10 (of 10 equal sized band excisions) of the in-gel digests, we observe glycodelin with between 1 and 4 unique sequence matches.
3. Increases in this protein in blood are consistently linked to breast cancer, lung cancer, endometrial cancer, preeclampsia.
4. In addition, deficiency in this protein (in blood) leads to infertility. This represents an additional disorder we can look for in a clinical population at the same time.
5. Glycodelin was positively identified from the peptide mapping experiments (LC-MS/MS). In addition, post-translational modifications have been reported.
Based on the foregoing, the analysis of Glycodelin in one or more menstrual samples is believed to provide useful clinical data that may be useful for the patient to monitor and provide to their physician to evaluate the health of the patient based on a single or multiple samples to shown the current Glycodelin levels as well as the trend of Glycodelin levels over time.

In some of our embodiments, our intention was to identify a target protein, one that possibly plays a role as a cancer biomarker. We have identified GRP-78 as a cancer biomarker in menstrual fluid after reviewing tryptic digests and MS/MS analysis of peptides from the first 7 samples. In some of the menstrual blood samples we tested, we detect GRP-78. Of the proteins we have consistently detected, GRP-78 is our best target to investigate further for several reasons:

1. We observe it in 2 out of 7 of our tryptic digest analyses of 1 patient. And we see it consistently and repeatedly in patient 333 in multiple samples at different time points.
2. In excised band 7 (of 10 equal sized band excisions) of the center portion of the in-gel digest, we observe GRP-78 with 9 unique sequence matches.
3. Increases in this protein in blood are consistently linked to breast cancer, endometrial cancer, familial breast and ovarian cancer and pre-eclampsia.
4. In addition, mutation in this protein (in blood) may lead to colon cancer, HCC, glial tumors, gastric carcinoma and colorectal cancer.
5. 78 kDa glucose-regulated protein (GRP-78) was positively identified from the peptide mapping experiments (LC-MS/MS).
Based on the foregoing, the analysis of GRP-78 in one or more menstrual samples is believed to provide useful clinical data that may be useful for the patient to monitor and provide to their physician to evaluate the health of the patient based on a single or multiple samples to shown the current GRP-78 levels as well as the trend of GRP-78 levels over time.

In various embodiments, the evaluation is of a Lon protease homolog 2, peroxisomal as a Cancer Biomarker. We have identified Lon protease homolog 2, peroxisomal as a Cancer Biomarker in menstrual fluid after reviewing tryptic digests and MS/MS analysis of peptides from the first 7 samples. There are many and various groups of proteins in varied studies that are seen in Cancer patients to be present at higher or lower concentrations than the baseline (healthy) patient. Nonetheless, Lon protease homolog 2, peroxisomal is consistently correlated. In some of the menstrual blood samples we tested, we detect Lon protease homolog 2, peroxisomal. Of the proteins we have detected, Lon protease homolog 2, peroxisomal is a target to investigate further for several reasons:

1. We observe it in 4 out of 7 of our tryptic digest analyses from 3 patients. And we see it consistently and repeatedly in a patient in multiple samples at different time points.
2. In excised bands 3, 5, and 7 (of 10 equal sized band excisions) of both the center and blade portions of the in-gel digest, we observe Lon protease homolog 2, peroxisomal with 1 high-confidence, unique sequence match.
3. Increases in this protein in blood are consistently linked to Cancer in several papers.
4. In addition, deficiency, etc. (via mutation, etc.) in this protein (in blood) leads to other diseases. These represent additional disorders we can monitor in a clinical population at the same time as a marker for aging and mitochondrial dysregulation.
Based on the foregoing, the analysis of Lon protease homolog 2, peroxisomal in one or more menstrual samples is believed to provide useful clinical data that may be useful for the patient to monitor and provide to their physician to evaluate the health of the patient based on a single sample or multiple samples to shown the current Lon protease homolog 2, peroxisomal levels as well as the trend of Lon protease homolog 2, peroxisomal levels over time.

Our intention was also to identify a target protein, one that possibly plays a role as a cancer biomarker. We have identified peroxiredoxin-2 as a cancer biomarker in menstrual fluid after reviewing tryptic digests and MS/MS analysis of peptides from our first 7 samples. There are many and various groups of proteins in varied studies that are seen in cancer patients to be present at higher or lower concentrations than the baseline (healthy) patient. Nonetheless, peroxiredoxin-2 is consistently correlated. In some of the menstrual blood samples we tested, we detect peroxiredoxin-2. Of the proteins we have consistently detected, peroxiredoxin-2 is a good target as a cancer biomarker to investigate further for several reasons:

1. This is typically a low-abundance protein. Pathology is associated with increases that would correlate to detectable levels for our analysis. We are able to observe this protein at detectable levels in 2 out of 7 of our analyses of 2 patients.
2. In excised band 10 (of 10 equal sized band excisions) of the center portions of the in-gel digest, we observe peroxiredoxin-2 with 4 unique sequence matches.
3. Increases in this protein in blood are consistently linked cervical carcinoma, colorectal cancer, and hepatocellular carcinoma.
4. In addition, mutation in this protein (in blood) leads to oxidative stress. Increases are also linked to pterygium.
Based on the foregoing, the analysis of peroxiredoxin-2 in one or more menstrual samples is believed to provide useful clinical data that may be useful for the patient to monitor and provide to their physician to evaluate the health of the patient based on a single sample or multiple samples to shown the current peroxiredoxin-2 levels as well as the trend of peroxiredoxin-2 levels over time.

Our intention was also to identify a target protein associated with breast cancer, one that possibly plays a role as a biomarker. We have identified phosphatidylinositol 4-phosphate 5-kinase type-1 alpha (PIP5K1a) as a breast cancer biomarker in menstrual fluid after reviewing tryptic digests and MS/MS analysis of peptides from the first 7 samples. There are many and various groups of proteins in varied studies that are seen in cancer patients to be present at higher or lower concentrations than the baseline (healthy) patient. Nonetheless, PIP5K1a is shown to be conspicuously correlated in several studies. In some of the menstrual blood samples we tested, we detect PIP5K1a. Of the proteins we have consistently detected, PIP5K1a is our best target to investigate further for breast cancer correlation for several reasons:

1. We observe it in 2 of our tryptic digest analyses in 2 different patients.
2. In excised bands B9 and C10 (of 10 equal sized band excisions) of the in-gel digests, we observe PIP5K1a with 1 unique sequence match. However, both unique sequence matches were identical in the two different patient samples.
3. Increases in this protein in blood increase the risk of cancer because it contributes to cancer cell proliferation, survival, and invasion. This protein also has regulatory effects on KIF2A which is associated with neural diseases.
4. The PIP5K1a has been positively identified from the peptide mapping experiments (LC-MS/MS). Our intention was to identify a target protein associated with breast cancer, one that possibly plays a role as a biomarker. We have identified phosphatidylinositol 4-phosphate 5-kinase type-1 alpha (PIP5K1a) as a breast cancer biomarker in menstrual fluid after reviewing tryptic digests and MS/MS analysis of peptides from the first 7 samples.
Based on the foregoing, the analysis of PIP5K1a in one or more menstrual samples is believed to provide useful clinical data that may be useful for the patient to monitor and provide to their physician to evaluate the health of the patient based on a single sample or multiple samples to shown the current PIP5K1a levels as well as the trend of PIP5K1a levels over time.

Our intention was also to identify another target protein, one that possibly plays a role as a breast cancer biomarker. We have identified serotransferrin as a breast cancer biomarker in menstrual fluid after reviewing tryptic digests and MS/MS analysis of peptides from the first 7 samples. There are many and various groups of proteins in varied studies that are seen in breast cancer patients to be present at higher or lower concentrations than the baseline (healthy) patient. Nonetheless, serotransferrin is consistently correlated. In some of the menstrual blood samples we tested, we detect serotransferrin. Of the proteins we have consistently detected, serotransferrin is our best target to investigate further for several reasons:

1. We observe it in 4 out of 7 of our tryptic digest analyses, and we see it consistently and repeatedly in at least one patient in multiple samples at different time points.
2. In excised bands 2, 3, 5, 6, 7, and 8 (of 10 equal sized band excisions) of both the center and blade portions of the in-gel digest, we observe serotransferrin with from 10 to 14 unique sequence matches.
3. Increases in this protein in blood are consistently linked to breast cancer in several papers.
Based on the foregoing, the analysis of serotransferrin in one or more menstrual samples is believed to provide useful clinical data that may be useful for the patient to monitor and provide to their physician to evaluate the health of the patient based on a single sample or multiple samples to shown the current serotransferrin levels as well as the trend of serotransferrin levels over time.

Our intention was to identify a target protein, one that possibly plays a role as a cancer biomarker. We have identified Protein TANC1 as a cancer biomarker in menstrual fluid after reviewing tryptic digests and MS/MS analysis of peptides from the first 7 samples. There are many and various groups of proteins in varied studies that are seen in cancer patients to be present at higher or lower concentrations than the baseline (healthy) patient. Nonetheless, Protein TANC1 is consistently correlated. In several of the menstrual blood samples we tested, we detect Protein TANC1. Of the proteins we have consistently detected, Protein TANC1 is a good target to investigate further for several reasons:

1. We observe it in 4 out of 7 of our tryptic digest analyses in 3 patient samples. And we see it consistently and repeatedly in at least one patient in multiple samples at different time points.
2. In excised band 2, 3, 9, and 10 (of 10 equal sized band excisions) of both the center and blade portions of the in-gel digest, we observe Protein TANC1 with from 1 to 3 unique sequence matches.
3. Increases in this protein in blood are consistently linked to rhabdomyosarcoma, ovarian cancer and cervical cancer.
Based on the foregoing, the analysis of Protein TANC1 in one or more menstrual samples is believed to provide useful clinical data that may be useful for the patient to monitor and provide to their physician to evaluate the health of the patient based on a single sample or multiple samples to shown the current Protein TANC1 levels as well as the trend of Protein TANC1 levels over time.

Our intention was also to identify a target protein, one that possibly plays a role as a biomarker for disease. We have identified the Teashirt homolog 2 as a Alzheimer's Disease and breast cancer biomarker in menstrual fluid after reviewing tryptic digests and MS/MS analysis of peptides from the first 7 samples. There are many and various groups of proteins in varied studies that are seen in cancer and/or AD patients to be present at higher or lower concentrations than the baseline (healthy) patient. Nonetheless, the Teashirt homolog 2 is consistently correlated. In some of the menstrual blood samples we tested, we detect the Teashirt homolog 2. Of the proteins we have consistently detected, the Teashirt homolog 2 is a good target to investigate further for several reasons:

1. We observe it in 2 of our tryptic digest analyses.
2. In excised band 10 (of 10 equal sized band excisions) of the in-gel digests, we observe this protein with between 1 and 4 unique sequence matches.
3. Deficiency, etc. (via mutation, etc.) in this protein (in blood) are associated with Alzheimer's Disease, mammary tumorigenesis, and breast cancer.
Based on the foregoing, the analysis of Teashirt homolog 2 in one or more menstrual samples is believed to provide useful clinical data that may be useful for the patient to monitor and provide to their physician to evaluate the health of the patient based on a single or multiple samples to shown the current Teashirt homolog 2 levels as well as the trend of Teashirt homolog 2 levels over time.

In some embodiments, the present methods allow for repeated evaluation of vitamin and/or mineral disease- or health-related biomarkers. Such repeated evaluation allows for early detection of one or more deficiencies or excesses that can be mitigated with nutritional supplements or dietary changes, for example. Further, such repeated monitoring is useful to prevent the effects of long term vitamin and/or mineral imbalance (e.g. calcium deficiencies leading to, for example, bone maladies such as osteoporosis and/or hypocalcemia).

In some embodiments, the biomarker is one or more disease- or health-related biomarkers measured in a blood gas test. For instance, the female subject may have pH evaluated repeatedly. Such evaluations may be used to detect an acid-base imbalance, such as can occur with kidney failure, heart failure, uncontrolled diabetes, and infections. pH may be used along with other tests, such as electrolytes to determine if an electrolyte imbalance is present, glucose to evaluate blood sugar concentrations, and BUN and creatinine tests to evaluate kidney function.

In some embodiments, the biomarker is C-reactive protein (CRP). This biomarker may be repeatedly evaluated to establish health information related to levels of inflammation, which is central to a number of diseases or disorders, including without limitation coronary heart disease, diabetes, macular degeneration, and cognitive decline. Measurement of CRP is predictive of a risk of incident myocardial infarction, stroke, peripheral arterial disease, and sudden cardiac death among healthy individuals with no history of cardiovascular disease, and predictive of recurrent events and death in patients with acute or stable coronary syndromes. Further, increased levels of C-reactive protein have been strongly linked with a greater risk of developing type II diabetes. Reliable and early detection of rising CRP can allow for appropriate intervention with diet, supplements, or anti-inflammatory therapy before onset significant health detriments. Illustrative lifestyle changes which may be directed by observed increases in CRP include the use of one or more of omega-3 supplements (e.g. fish oil, krill oil, etc.), L-carnitine, and soluble fiber before meals.

In some embodiments, the biomarker is fibrinogen, which plays a role in blood clotting and increases in response to tissue inflammation. Since the development of atherosclerosis and heart disease are essentially inflammatory processes, increased fibrinogen levels can help predict the risk of heart disease and stroke. High fibrinogen levels not only are associated with an increased risk of heart attack, but also are seen in other inflammatory disorders such as rheumatoid arthritis and glomerulonephritis. A repeated evaluation of fibrinogen levels helps prevent or mitigate any of these diseases or disorders. A combination of lifestyle and behavioral changes—such as quitting smoking, losing weight, and becoming more physically active—may be directed by the present monitoring. Further, increases in fibrinogen may direct one or more nutritional interventions, such as omega-3 supplements (e.g. fish oil, krill oil, etc.), niacin, and folic acid, and vitamins A and C.

In some embodiments, the biomarker is dehydroepiandrosterone (DHEA), a hormone produced by the adrenal glands, which is a precursor to the sex hormones estrogen and testosterone. Blood levels of DHEA peak in one's twenties and then decline dramatically with age, decreasing to 20-30% of peak levels between the ages of 70 and 80. DHEA is frequently referred to as an “anti-aging” hormone. Healthy levels of OHEA may support immune function, bone density, mood, libido, and healthy body composition. Elevated levels of DHEA may indicate congenital adrenal hyperplasia, a group of disorders that result from the impaired ability of the adrenal glands to produce glucocorticoids. Supplementation with DHEA increases immunological function, improves bone mineral density, increases sexual libido in women, reduces abdominal fat, protects the brain following nerve injury, and helps prevent diabetes, cancer, and heart disease. Natural therapies may help to optimize DHEA levels, e.g. pregnenolone or DHEA. Accordingly, DHEA is a biomarker for which repeated evaluation is beneficial.

In some embodiments, the biomarker is thyroid stimulating hormone (TSH), which controls thyroid hormone secretion in the thyroid. When blood levels fall below normal, this indicates hyperthyroidism (also called thyrotoxicosis), and when values are above normal, this suggests hypothyroidism. Overt hyper- or hypothyroidism is generally easy to diagnose, but subclinical disease can be more elusive and therefore repeated evaluation is beneficial. Further, because the symptoms of thyroid imbalance may be nonspecific or absent and may progress slowly, and since many doctors do not routinely screen for thyroid function, mild hyper- or hypothyroidism can go undiagnosed for some time. Undiagnosed mild disease can progress to clinical disease states. Mild hypothyroidism (low thyroid gland function) may be associated with reversible hypercholesterolemia (high blood cholesterol) and cognitive dysfunction, as well as such nonspecific symptoms as fatigue, depression, cold intolerance, dry skin, constipation, and weight gain. Mild hyperthyroidism is often associated with atrial fibrillation (a disturbance of heart rhythm), reduced bone mineral density, and nonspecific symptoms such as fatigue, weight loss, heat intolerance, nervousness, insomnia, muscle weakness, shortness of breath, and heart palpitations. Accordingly, the use of the present methods may allow for early detection to avoid or mitigate diseases or disorders related to TSH imbalance. In some embodiments, further supplementation with one or more of L-tyrosine, iodine, and selenium may be directed by the present methods.

In some embodiments, the biomarker is homocysteine. High homocysteine levels have been associated with increased risk of heart attack, bone fracture, and poor cognitive function. Further, incremental increases in the level of homocysteine correlate with an increased risk for coronary artery disease, indicating a benefit of repeated evaluation. Homocysteine has also become recognized as an independent risk factor for bone fractures. The present methods may inform the use of vitamin B12, vitamin 85, folic acid, and trimethylglycine to optimize homocysteine levels.

In some embodiments, the biomarker is a blood ketone. This biomarker may be useful to monitor the development of monitor diabetic ketoacidosis (OKA) in female subjects with type 1 and sometimes type 2 diabetes. OKA is associated with acute hyperglycemia, a severe insulin deficiency, and a disruption of the body's acid-base balance.

In some embodiments, the biomarker is relevant to cancer diagnosis, prognosis or treatment response. For example, a female subject may be repeatedly screened for one or more known cancer biomarker.

For example, in some embodiments, the biomarker is CEA. In some embodiments, a rise in CEA over the course of periodic evaluations is indicative of a disease or disorder. Such a biomarker may be repeatedly evaluated in instances in which a female subject may be afflicted with various cancers. For example, such subjects may have a family history of these cancers, be a cancer survivor that is testing for recurrence, etc. CEA may be monitored for colorectal, pancreatic, lung, breast, ovarian, urinary tract, medullary thyroid or other cancers. Further, a rise in CEA may be indicative of RA, hepatitis, COPO, colitis, pancreatitis, inflammation, cirrhosis, peptic ulcer, ulcerative colitis, rectal polyps, emphysema, benign breast disease.

This biomarker is illustrative of a need for repeated evaluation. For example, the levels of CEA increase in certain conditions and/or lifestyle choices and thus run the risk of false positive data (and, in turn, possible unnecessary further testing or treatment). By way of non-limiting example, CEA levels may be obfuscated by one or more of cigarette smoking, liver and gallbladder problems (e.g. cirrhosis and/or cholecystitis), inflammatory bowel diseases (such as ulcerative colitis or diverticulitis), lung infection(s), inflammation of the pancreas (pancreatitis) and stomach ulcer. For instance, the normal range of CEA is about 0-2.5 mcg/L, while in cigarette smokers the normal range is about 0-5 mcg/L. The present methods establish a baseline of biomarker levels that correct for biomarker aberrations related to certain conditions and/or lifestyle choices. In this example, repeated testing would correct for CEA elevation associated with smoking. For instance, if a smoker has a CEA test, the repeated evaluation of the present methods would allow for a health practitioner to note that a high value is really the smoker's baseline value.

In some embodiments, the female subject may repeatedly be evaluated for one or more cancer markers to which the female subject is susceptible. For example, the female subject may have a family medical history which includes one or more hereditary cancers, such as breast cancer, colorectal cancer, ovarian cancer, pancreatic cancer. stomach cancer, and uterine cancer. Further, the female subject may be a cancer survivor who is repeatedly evaluated for one or more disease- or health-related biomarkers for the early detection of recurrence.

In various embodiments, the female subject is repeatedly evaluated for one or more disease- or health-related biomarkers associated with breast cancer. For instance, such a female subject may be one who previously had breast cancer and/or has a family history of breast cancer. For instance, the female subject may apply the methods described herein to monitor for breast cancer recurrence. Illustrative disease- or health-related biomarkers to be evaluated include one or more of estrogen receptor (ER), progesterone receptor (PR). Such evaluation is indicative of, if afflicted with breast cancer, a likelihood of response to one or more hormone therapies, such as tamoxifen (NOLVADEX), the presence of the disease- or health-related biomarkers indicating a higher likelihood of response. Another biomarker of interest, especially in the context of breast cancer is human epidermal growth factor receptor 2 ((HER2), which is indicative of, if afflicted with breast cancer, a likelihood of response to anti-HER2 treatments, such as trastuzumab (HERCEPTIN), and in some cases, may suggest whether additional treatment with chemotherapy may be helpful. Another biomarker of interest, especially in the context of breast cancer is one or more of cancer antigen 15-3 (CA 15-3), cancer antigen 27.29 (CA27.29), and carcinoembryonic antigen (CEA). These disease- or health-related biomarkers are particularly informative of an occurrence of metastatic cancer but may also be helpful in the diagnosis and/or prognosis of, for example, inflammation, cirrhosis, peptic ulcer, ulcerative colitis, rectal polyps, emphysema, and benign breast disease. Yet another biomarker of interest, especially in the context of breast cancer is one or more of urokinase plasminogen activator (uPA) and plasminogen activator inhibitor (PA1-1). Higher-than-normal levels of these tumor markers in the cancer tissue may mean that the cancer is more aggressive (e.g. faster growing). Further, these tumor markers may be used to guide the use of chemotherapy after surgery for patients with node-negative breast cancer.

In various embodiments, the female subject is repeatedly evaluated for one or more disease- or health-related biomarkers associated with colon or colorectal cancer. For instance, such a female subject may be one who previously had colon or colorectal cancer and/or has a family history of colon or colorectal cancer. For instance, the female subject may apply the methods described herein to monitor for colon cancer recurrence. Illustrative disease- or health-related biomarkers include those described in Mo/ Diagn Ther. 2011 Jun. 1; 15(3):129-41 or World J Gastrointest Oneal 2014 Apr. 15; 6(4): 83-97, the contents of which are hereby incorporated by reference in their entirety. In some embodiments, the biomarker is methylated Septin 9 DNA (mSEPT9), and an increase in this biomarker is indicative or an occurrence of high likelihood of occurrence of colon cancer. In some embodiments, including those testing RNA, the biomarker is one or more of ANXA3, CLEC4D, LMNB1, PRRG4, TNFAIP6, AND VN1, the overexpression of which is indicative or an occurrence of high likelihood of occurrence of colon cancer and IL2RB the reduced expression of which is indicative or an occurrence of high likelihood of occurrence of colon cancer.

In various embodiments, the female subject is repeatedly evaluated for one or more disease- or health-related biomarkers associated with ovarian cancer. For instance, such a female subject may be one who previously had ovarian cancer and/or has a family history of ovarian cancer. For instance, the female subject may apply the methods described herein to monitor for ovarian cancer recurrence. In many women with ovarian cancer, levels of CA-125 are high and therefore this biomarker is included in the present methods. In various embodiments, the biomarker for ovarian cancer of the present methods is one or more of Table 2 of Cortesi et al. Electrophoresis 2011, 32, 1-12, the entire contents of which are hereby incorporated by reference. For instance, the biomarker for ovarian cancer of the present methods may be one or more Retinoblastoma-binding protein 4, Elongation factor I-a 1, Malate dehydrogenase mitochondrial, Glyceraldehyde-3-phosphate dehydrogenase, Osteoglycin, Annexin 5, Hydroxyacyl-coenzyme A dehydrogenase mitochondrial, proteasome activator complex subunit 2, Galectin-3, Calcium-activated neutral proteinase small subunit 1, Glutathione-S-transferase Mu-3, Peroxiredoxin-6, Triosephosphate isomerase, Adenylatekinase 3, Tumor protein D52, Rho GDP dissociation inhibitor 1, Apolipoprotein A-I, Serum amyloid P-component, Glutathione-S-transferase Mu2, Glutathione-S-transferase Mu1, Glutathione-S-transferase Mu1, Flavin reductase, Peroxiredoxin-1, Cleavage and polyadenilation specificity factor 5 subunit Glutathione S-transferase A2, Adenylate kinase isoenzyme 1, Transgelin, Translationally-controlled tumor protein, Lactoylglutathione lyase, Synthase subunit d, mitochondrial, Ubiquitin-conjugatin enzyme E2 K, Glutathione-S-transferase P1, Abhydrolase domain-containing protein 14B, Phosphatidylethanolamine-binding protein 1, Peptidyl-prolyl cis-trans isomerase B, Heat shock protein b 6, Cytochrome b5, Eukariotic translation initiation factor 5A-1, Transthyretin, Ubiquitin-conjugatin enzyme E2 N Retinal binding protein, Galectin-1, Hemoglobin subunit b, Hemoglobin subunit b, Profilin 1 Hemoglobin subunit a, Protein S100-A8-calgranulin A, Protein S100-A8-calgranulin A, b-2 microglobulin Histone H4, Protein S100-A6 Peroxiredoxin-1, Ubiquitin, Superoxide dismutase, Heat shock protein b1, Abhydrolase domain containing protein 11 GTP-binding nuclear protein Ran, and Superoxide dismutase (Mn) mitochondrial. In various embodiments, the biomarker for ovarian cancer of the present methods is one or more of Tables 4 or 5 of Gynecologic Oncology 108 (2008) 402-408, the entire contents of which are hereby incorporated by reference,

In various embodiments, the female subject is repeatedly evaluated for one or more disease- or health-related biomarkers associated with pancreatic cancer. For instance, the female subject may apply the methods described herein to monitor for pancreatic cancer recurrence. For instance, such a female subject may be one who previously had pancreatic cancer and/or has a family history of pancreatic cancer. For instance, the female subject may have a CA 19-9 (Cancer antigen 19), as part of the repeated evaluation. Further, CEA (Carcinoembryonic antigen) may be monitored. Further, elevation of amylase over time may be indicative of pancreatic cancer.

In various embodiments, the female subject is repeatedly evaluated for one or more disease- or health-related biomarkers associated with lung cancer. For instance, such a female subject may be one who previously had lung cancer and/or has a family history of lung cancer. For instance, the female subject may apply the methods described herein to monitor for lung cancer recurrence.

In some embodiments, the biomarker is a matrix metalloproteinase such as matrix metalloproteinase-2 (MMP-2), -9 (MMP-9), and -13 (MMP-13) and the cancer is colorectal and/or bladder cancer.

In some embodiments, the biomarker is circulating tumor DNA (ctDNA), namely, genome fragments that float freely through the bloodstream.

In various embodiments, the female subject is repeatedly evaluated for one or more disease- or health-related biomarkers associated with endometriosis. Endometriosis is a gynecological disease defined as the presence of endometrial tissue outside the uterine cavity. This tissue is located in the peritoneum, ovary or fallopian tube and more rarely in the pleura, lung or brain. Endometriosis occurs in 5-20% of females with pelvic pain, 20-50% of infertile females and 6-10% of females of reproductive age. The causes of this disease include, among others, retrograde menstruation, endometrium abnormalities, peritoneal environment changes, increased angiogenesis, inadequate immunological reactions and genetic and environmental factors. In various embodiments, the present invention relates to the evaluation of endometriosis, for instance by measuring one or more of annexin V, VEGF, CA-125, slCAM-1/or glycodelin, MIF, CD74, IL-6, IL-8 and COX-2 may be evaluated using the samples and methods of the present invention)(see, e.g., Hum Reprod. 2012 September; 27(9):2698-711, Fertil Steril. 2015 January; 103(1):153-9.e3, Hum Reprod. 2010 March; 25(3):654-64, the entire contents of which are hereby incorporated by reference). In various embodiments, the present invention relates to the evaluation of endometriosis, for instance by measuring one or more of octamer-binding transcription factor 4 (Oct-4), C-X-C chemokine receptor type 4 (CXCR4), SRY-box containing gene 2 (SOX2) and mesenchymal-epithelial transition factor (MET), collapsin response mediator protein 2 (CRMP2), ubiquitin carboxyl-terminal hydrolase isozyme L1 (UCH-L1) and myosin regulatory light polypeptide 9 (MYL9 may be evaluated using the samples and methods of the present invention)(see, e.g., Molecular Medicine Reports 8: 183-188, 2013, the entire contents of which are hereby incorporated by reference).

In some embodiments, a female subject is evaluated for a variety of disease- or health-related biomarkers that relate to delusion or hallucination. For example, a female subject with a family history of psychiatric disorders or diseases. For example, one or more disease- or health-related biomarkers found in, for example, Table 5A, Table 5B, Table 6A, and Table 6B of US Patent Publication No. 2011/0098188, the contents of which are hereby incorporated by reference in their entirety, are useful. In some embodiments, the genes Drd2, ApoE, Nab1, ldh1, Scamp1, Ncoa2, Aldh111, Gpm6b are evaluated and a decrease in expression is indicative of a higher likelihood of high delusions states or the genes Nrg1, Egr1, Dctn1, Nmt1, Pllp, Pvalb, Nmt1, Pctk1 are evaluated and an increase in expression is indicative of a higher likelihood of high delusions states. Accordingly, the repeated evaluation may direct the administration of anti-psychotic agents as known in the art.

In some embodiments, the biomarker is the brain protein tau. This biomarker may be used as an indicator of brain injuries, for example, concussions. For example, the female subject may be an athlete that monitors brain status to avoid long term complications associated with concussions (e.g. memory problems, lack of inhibition, intense anger and/or aggression, personality changes, inattention and lack of concentration, problems organizing, planning, and problem solving, and language impairment).

In some embodiments, the biomarker is one that is informative for heart health, such as one or more troponins (e.g. a cardiac-specific troponin I or troponin T test), CK-MB, and myoglobin.

In various embodiments, the present methods relate to monitoring for signs of Alzheimer's disease. For instance, the female subject may have relatives with Alzheimer's disease and may monitor Alzheimer's blood markers repeatedly, including for example, IRS-1 and tau.

Liver damage, including liver fibrosis and cirrhosis, may be monitored with the present methods. For example, aspartate transaminase and alanine transaminase may be measured as disease- or health-related biomarkers. For example, a AST/ALT ratio, the ratio between the concentrations of aspartate transaminase (AST)(aspartate aminotransferase) and alanine transaminase (ALT)(alanine aminotransferase), in the blood is useful to differentiate between causes of liver damage, or hepatotoxicity. Further, blood cell monitoring, including complete blood counting, may be indicative of liver function.

Further, in some embodiments, the biomarker is alpha-fetoprotein (AFP) and is useful for long term evaluation of liver diseases or disorders (e.g. hepatitis). Increases in AFP are associated with hepatocellular carcinoma, germ cell tumors, and metastatic cancers of the liver.

In various embodiments, the biomarker is one or more antibodies that may, for example, reflect an infection. For example, lgA, lgD, lgE, lgG and lgM, may be measured over time and increases may be indicative of increased immunological activity. For example, if a female subject is suspected of having Lyme disease and shows increases in lgM and/or lgG, that rise over time, then it is likely that the person has an active B. burgdorferi infection. Further antibodies can be used to indicate affliction with one or more of Multiple myeloma and Waldenström macroglobulinemia.

In various embodiments, the biomarker is one or more steroids. For example, in some embodiments, the biomarker is cortisol. Different diseases, such as Cushing syndrome and Addison disease, can lead to either too much or too little production of cortisol. Measuring blood cortisol level can help diagnose these conditions. It is also measured to evaluate how well the pituitary and adrenal glands are working. Further, cortisol may be used to measure long term stress and indicate lifestyle changes are necessary.

In various embodiments, the biomarker is one or more of the biomarkers listed in the table below. In some embodiments, the biomarker of the left-most column is useful in the evaluation of a subject, by way of non-limitation, by evaluation of menstrual fluid from the patient, for a disease in the column labelled “illustrative disease.” In various embodiments, the biomarker of the left-most column is used in the diagnosis, or prognosis, or evaluation of response to treatment of the illustrative disease. The references of the below table are hereby incorporated by reference in their entirety, especially as to descriptions linking the enumerated biomarker to the enumerated disease. In various embodiments, one or more (e.g. 1, or 2, or 3, or 4, or 5, or 6, etc.) of the illustrative biomarkers can be used in the evaluation of a patient for an illustrative disease. By way of non-limiting example, in some embodiments, evaluation of Activin A and follistatin can be used to evaluate endometrial function including the diseases below as well as dysfunctional uterine bleeding (see, e.g., Reprod Sci. 2007 May; 14(4):383-9, the entire contents of which are hereby incorporated by reference). In some embodiments, inhibin and activin is evaluated in the context of ovarian cancer e.g. as a measure in diagnosis and management and also as a factor in the pathogenesis of these tumors (see, e.g., Endocr Re/at Cancer. 2004 March; 11(1):35-49, the entire contents of which are hereby incorporated by reference). In another embodiment, follistatin (FST) and CA-125 can be used to evaluate ovarian cancer (and, by way of non-limitation, reduce the number of false-positive results in diagnosis) (see, e.g., J Int Med Res. 2012; 40(3):877-86, the entire contents of which are hereby incorporated by reference). Further, in some embodiments, follistatin (FST) and BRCA1 can be used to evaluate ovarian cancer and human ovarian surface epithelial cells (see, e.g., PLoS One. 2012; 7(6):e37697, the entire contents of which are hereby incorporated by reference). Further, in some embodiments, follistatin (FST) and activin A can be used to evaluate peritoneal, ovarian and deep infiltrating endometriosis (see, e.g., Human Reproduction, Vol. 00, No. 0 pp. 1-7, 2009 doi:10.1093/humrep/dep195, the entire contents of which are hereby incorporated by reference). In some embodiments, EMMPRIN and fascin may be used in the evaluation of ovarian cancer, including differential diagnosis of some diagnostically problematic mucinous ovarian tumors (see, e.g., Pathol Res Pract. 2014 December; 210(12):934-8, the entire contents of which are hereby incorporated by reference).

In various embodiments, the biomarker is follistatin. Follistatin (FST) is a monomeric glycoprotein that inhibits release of follicle-stimulating hormone from the pituitary. It is a specific binding protein of activin and is involved in the regulation of multiple physiological and pathological functions, and has important roles in early embryonic development, differentiation of ovarian granulosa cells, liver fibrosis and polycystic ovarian syndrome. FST's levels vary with physiological and pathological conditions such as pregnancy and cancer. Follistatin circulates in two major isoforms: a full-length molecule composed of 315 amino acids (FS315), and a short isoform of 288 amino acids (FS288) generated by alternative splicing of the Fst gene. The activin/follistatin system is thought to act primarily as a local growth regulator system controlling proliferation, differentiation and apoptosis of many cell types in an autocrine and paracrine manner. Of interest is the full expression of the activin/follistatin system in human endometrium. Activin A stimulates the decidualization of endometrial stromal cells and aberrant expression of the activin/follistatin axis has been observed in the endometria of women with recurrent miscarriage (Fertil Steril 2006; 86:1723-1730, the entire contents of which are hereby incorporated by reference), anovulatory bleeding (Reprod Sci 2007; 14:383-389, the entire contents of which are hereby incorporated by reference) and endometriosis (Aust N Z J Obstet Gynaecol 2006; 46:148-153, the entire contents of which are hereby incorporated by reference). In various embodiments, the present invention relates to the measurement of FST, e.g. in menstrual fluid, in the context of evaluating one or more of the diseases described herein.

In various embodiments, the biomarker is activin A The activins are a family of proteins which consist of disulphide-linked homodimers and heterodimers of the subunits of inhibin termed _{A} and s. These three proteins, called activin A (A-A), activin B (s-s) and activin AB (A-B), are members of the transforming growth factor (TGF) super-family of proteins. Although the activins were originally isolated for their ability to stimulate follicle-stimulating hormone secretion, they have been shown to influence many biological processes, including parenchymal haemopoiesis, embryogenesis, neurotransmission, hepatic parenchymal cell division, prostate biology and angiogenesis. In various embodiments, evaluation of cancers via activin, e.g. as described in Cancers 2015, 7, 70-91, the entire contents of which are hereby incorporated by reference, is provided. In various embodiments, the present invention relates to the measurement of activin A, e.g. in menstrual fluid, in the context of evaluating one or more of the diseases described herein.

In various embodiments, the biomarker is CA-125. CA-125 has found application as a tumor marker or biomarker that may be elevated in the blood of some patients with specific types of cancers, or other benign conditions. CA 125 is most consistently elevated in epithelial ovarian cancer, but can be expressed in a number of gynecologic (e.g. endometrial, fallopian tube) and non-gynecologic (pancreatic, breast, colon and lung) cancers. The best established application of the CA 125 assay is in monitoring ovarian cancer. The rate of decline in CA 125 during primary chemotherapy has been an important independent prognostic factor in several multivariate analyses. Persistent elevation of CA 125 at the time of a second look surgical surveillance procedure predicts residual disease with >95% specificity. Rising CA-125 values have preceded clinical detection of recurrent disease by at least 3 months in most, but not all studies. Rising CA 125 during subsequent chemotherapy has been associated with progressive disease in more than 90% of cases. In various embodiments, the present invention relates to the measurement of CA-125, e.g. in menstrual fluid, in the context of evaluating one or more of the diseases described herein.

In various embodiments, the biomarker is fascin. Fascin is an actin-bundling protein that has a major function in forming parallel actin bundles in cell protrusions such as lamellipodia, which are key specializations of the plasma membrane for cell migration. Fascin overexpression has been reported in many different types of carcinomas, including breast, ovary, colon, pancreas, esophagus, stomach, lung, and urinary bladder, as well as in other tumors, such as lymphomas, sarcomas, melanomas, and astrocytomas. The high expression of fascin is correlated with an aggressive clinical course and shorter survival. Fascin organizes actin into highly dynamic and architecturally diverse subcellular scaffolds. These scaffolds orchestrate a variety of mechanical processes, including filopodial protrusions in motile cells. In various embodiments, the present invention relates to the measurement of fascin, e.g. in menstrual fluid, in the context of evaluating one or more of the diseases described herein.

In some embodiments, the present invention relates to a device for collection of a female subject's menstrual fluid sample and uses thereof. In some embodiments, the device is a disposable cartridge which may be inserted into a wireless enabled device. In various embodiments, the device is a home instrument. In various embodiments, the device is operated by the patient, without the need for intervention by a medical professional. Accordingly, in various embodiments, the patient is spared the inconvenience of scheduling an appointment in a medical clinical and may be able to institute sample collection at her convenience and without scheduling delays.

In various embodiments, the device is or comprises a sampling implement that provides a means to collect a sample from a subject. The sampling implement may be connected to a collection chamber via a sampling implement holder. In some embodiments, the sampling implement is disposed at the distal end of a shaft, which shaft can be solid, hollow or semi-permeable. In some embodiments, the sampling implement is a swab, a comb, a brush, a spatula, a rod, a foam, a flocculated substrate or a spun substrate.

In various embodiments, the device is associated with and/or integrated into one or more of a tampon, pad (menstrual napkin) or menstrual cup (see, e.g., International Patent Publication Nos. WO/2002/080827 and WO/2006/058409, the contents of which are hereby incorporated by reference).

In various embodiments, the collection of menstrual fluid may take place on one of the heaviest days of the donor's menstrual period which may be the first or second day.

In various embodiments, a single sample or multiple samples may be collected. The sample or samples may be maintained at room temperature (about 15° C. to about 25° C.). In various embodiments, samples may be shipped to a laboratory so long as the sample or samples arrive at the laboratory within about 24 hours to about 72 hours of collection. Alternatively, samples may be refrigerated at about 1° C. to about 10° C.

In various embodiments, the sample may be subjected to centrifugation and either the supernatant or pellet may be analyzed.

In various embodiments, the evaluation comprises measuring a presence, absence, or level of a protein. In various embodiments, the evaluation comprises measuring a presence, absence, or level of expression of a nucleic acid.

In some embodiments, the present methods comprise contacting an agent that specifically binds a biomarker with the menstrual sample. For example, such an agent may be an antibody. Illustrative, but non-limiting methods for evaluation include one or more immunohistochemical staining, western blotting, in cell western, immunofluorescent staining, ELISA, and fluorescent activating cell sorting (FACS), or any other method described herein or known in the art.

There are generally two strategies used for detection of epitopes on antigens in body fluids or tissues, direct methods and indirect methods. The direct method comprises a one-step staining, and may involve a labeled antibody (e.g. FITC conjugated antiserum) reacting directly with the antigen in a body fluid or tissue sample. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et a. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, Calif.).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the VL, VH, CL and CH1 domains; a F(ab)2 fragment, a bivalent fragment including two Fab fragments linked by a disulfide bridge at the hinge region; a Fd fragment consisting of the VH and CH1 domains; a Fv fragment consisting of the VL and VH domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

In another embodiment, the measurement comprises evaluating a presence, absence, or level of a nucleic acid.

A person skilled in the art will appreciate that a number of methods can be used to detect or quantify the DNA/RNA levels of various disease- or health-related biomarkers.

Gene expression can be measured using, for example, low-to-mid-plex techniques, including but not limited to reporter gene assays, Northern blot, fluorescent in situ hybridization (FISH), and reverse transcription PCR(RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP.

A person skilled in the art will appreciate that a number of methods can be used to detect or quantify the level of RNA products of the disease- or health-related biomarkers within a sample, including arrays, such as microarrays, RT-PCR (including quantitative PCR), nuclease protection assays and Northern blot analyses.

In various embodiments, the present methods allow for efficient transfer of healthcare information between medical practitioners. For example, any of the methods and systems of US Patent Publications Nos. 2014/0164022, 2013/0060574, and 2007/0135690, the contents of which are hereby incorporated by reference in their entirety, may be used.

In some embodiments, the method provides a database of the female subject's biomarker information. In various embodiments, the database is locally or remotely stored. In various embodiments, the database is cloud-based. In various embodiments, the database can be stored and/or transferred via physical transport media, for example on a USB thumb drive, tablet or phone, CD ROM, or smart card, and/or via transport media, secure transmission can occur via a network line, or wireless cell phone communication, Internet, ultrasound, Bluetooth, or near-field communication.

In various embodiments, the database comprises a subject interface and/or a healthcare provider interface, the interfaces optionally being graphical user interfaces (GUIs). In various embodiments, the subject interface and/or a healthcare provider interface is accessible via an application on a personal communication device, optionally selected from a laptop computer, a tablet computer, a personal digital assistant (PDA), and a smart phone. Personal communication device include, for example, IPHONES (available from Apple Inc., Cupertino, Calif.), BLACKBERRY devices (available from Research in Motion, Waterloo, Ontario, Canada), or any smart phones equipped with the ANDROID platform (available from Google Inc., Mountain View, Calif.), tablets, such as the IPAD and KINDLE FIRE, and personal digital assistants (PDAs).

In various embodiments, the healthcare provider may be the female subject's physician or female subject's physician's associates and/or the healthcare provider may be personnel from laboratory that processes the female subject's menstrual fluid sample. In various embodiments, data flows to and from the database, for example, via wireless cell phone communication, Internet, ultrasound, Bluetooth, or near-field communication.

In various embodiments, the database is protected by automatic logoff, which is required by HIPAA Either the subject-side software or the wireless LAN or both will initiate automatic logoff to disconnect a user after a pre-configured period of time of inactivity.

In various embodiments, the database not only stores the female subject's biomarker information but also provides suggestions of lifestyle changes based on such information. These suggestions may be entered by a healthcare provider or automatically generated from medical databases (e.g. via wireless cell phone communication, Internet, ultrasound, Bluetooth, or near-field communication, such as WEBMD). For example, the database may deliver such suggestions to the female subject via an application on a personal communication device. For example, repeated evaluation of calcium may show a decrease over time and the database may generate a suggestion to the female subject to effect one or more lifestyle changes (e.g. administer a calcium supplement, increase intake of diary in the diet, etc.).

In some embodiments, the female subject utilizes the subject interface to access information stored on the database. In some embodiments, the healthcare provider utilizes the healthcare provider interface to access information stored on the database. In some embodiments, various different healthcare providers utilize the healthcare provider interface to access information stored on the database. For example, in some embodiments, 2 or more, or 3 or more, or 4 or more different healthcare providers utilize the healthcare provider interface to access information stored on the database. Such different healthcare providers may be specialists that communicate with the subject and/or the subject's primary different healthcare providers, such specialists including, for example, oncologists, rheumatologists, etc.

In some embodiments, other parties, including health and/or life insurance providers, may utilize the healthcare provider interface to access information stored on the database. For example, health and/or life insurance providers may be provided access to assess insurance eligibility and/or to allow for reduce premiums by de-risking a female subject's insurance via monitoring (e.g. a female subject providing access to the repeated evaluations provided herein may receive lower premiums). For example, data can be transferred to such insurance providers in lieu of physical testing. Also, proof of regular monitoring may be used to assess insurability of a female subject.

In various embodiments, the present methods and/or any evaluation/database of female subject information is used in a healthcare system (e.g. with some of the insurance features described herein) to create a rewards program to offer incentive for women to take monthly samples. For example, a health insurance may monetarily incentivize sample evaluation by offering rebates.

In various embodiments, the present methods and/or any evaluation/database of female subject information is used to disseminate information across patient communities committed to finding cures, including patient support groups and disease-specific organizations/foundations (e.g. American Cancer Society, Komen, Alzheimer's Foundation of America, etc.). In some embodiments, the present methods create disease databases that allow for development of more effective therapeutic options in any of the diseases described herein.

In various embodiments, the present methods and/or any evaluation/database of female subject information is combined with self-measurement of physiological parameters to provide further information of health status, for example, one or more of heart rate, blood pressure, number of steps walked, quality of sleep, calories consumed, and calories burned. In some embodiments, the present methods further comprises evaluating one or more of a pain score, allergies, mood, food/dietary information, health checklists, healthcare records, medications, tests, test results, care plans, and discharge plans. In some embodiments, the present methods are combined with data from activity tracking devices (e.g. FITBIT, Jawbone UP, Nike+ FuelBand, etc.). In some embodiments, the present methods are used in biometric analysis of an athlete. For example, in some embodiments, an athlete may use the present methods to track the progress of recovery from a long-term injury (e.g. a sprain, bone breakage, etc., which may use, by way of non-limiting example, CRP evaluation).

In some embodiments, the database is suitable for database warehousing. In some embodiments, the database is integrated into the female subject's existing electronic medical records. For example, when appropriate the database may be linked with the subject's genetic data/genetic information such as, for example, the sort that may be generated in an oncology patient. In various embodiments, the database is used to create a Continuity of Care Record (CCR). The present invention provides for adding data to the CCR via the present methods and transmitting the data and edited CCR via wireless cell phone communication, Internet, ultrasound, Bluetooth, or near-field communication (e.g. using the database of the present disclosure).

In various embodiments, the present methods allow for improved research and understanding of women's health. For instance, in some embodiments, the present methods improve epidemiological analysis of women's health (e.g. analysis of diseases specific to women, analysis of diseases that progress differently in women than men). Further, in various embodiments, the present methods allow for studies of how women react to therapeutic agents (e.g. in the clinical trial setting, e.g. allowing study of pharmacodynamics and pharmacokinetic parameters of certain agents with females). Accordingly, in some embodiments, the present methods allow for more efficient clinical trial design that is cognizant of gender differences.

In various embodiments, the female subject is menstruating and thus generating sample for evaluation. In some embodiments, the female subject is non-menopausal or recently menopausal. In some embodiments, the female subject may be repeatedly evaluated until pregnancy and resume evaluation post-partum. In these embodiments, the female subject may be monitored for post-partum complications. For instance, the pre-pregnancy data may be used in comparison with post-partum data to monitor a restoration of pre-pregnancy health baselines.

In some embodiments, the female subject has an age in a range of from about 13 years to about 60 years. In some embodiments, the female subject is about 10 years old, or about 15 years old, or about 20 years old, or about 25 years old, or about 30 years old, or about 35 years old, or about 40 years old, or about 45 years old, or about 50 years old, or about 55 years old, or about 60 years old, or about 65 years old. The collection of multiple menstrual blood samples may be used provide a long term graphic or health history for various biomarkers of interest and may be used to provide an outlook for the health of the patient when they are post-menopausal. As part of this, a risk scoring method may be used or developed to identify higher risk patients based on the previously collected biomarker levels.

The women's reproductive system is an active environment composed of multiple structures working together. Although each structure is responsible for its own unique function, the systems functions are mediated each month from puberty until menopause by different stages of the menstrual cycle. The process of menstruation occurs for approximately 3-5 days at the beginning of each monthly cycle. The follicles present at the distal tubal opening of the fallopian tube generate a flux of menstrual fluids and mucosal tissue layers throughout the cycle that are ultimately shed with the endometrial lining during menstruation, and secreted as menstrual blood through the cervix, out of the vagina and ultimately discarded. Throughout much of history, menstruation has been accepted as an innate and necessary function of the female reproductive system with little inquiry into the proteomic constituents of menstrual blood native to the females reproductive system. However, in recent years, what we have come to understand about menstrual secretions has changed dramatically. With the accumulation of proteins and cellular debris throughout the menstrual cycle, studies have shown that menstrual blood actually contains a variety of proteins that have promising potential to provide insight into the gynecologic state of the patient. The identification of proteomic biomarkers in menstrual blood offers a unique opportunity to bypass the current limitations in diagnosing gynecologic malignancies such as ovarian cancer by exploiting these monthly secretions during menstruation to evaluate the gynecologic state of both healthy and diseased individuals. As a result of the shared circulation between systemic circulation and reproductive health, other biomarkers can be found within menstrual blood that are indicative of general well-being and health outside of the female reproductive system.

A purpose of this Example is to deliver a point-of-care diagnostic tool to women from “bench to bedside”. With the progress of technology and proteomic analysis comes the opportunity to develop tools accurate and effective enough to replace the current and ineffective diagnostic protocols that use biomarkers to screen for malignancies at the proximal level of the reproductive system, in addition to indications of malignancies throughout the rest of the bod and/or the general well-being of the female.

The Example in phase I focuses on the validation of the correlation of biomarkers found in menstrual blood to biomarkers in venous circulation, important because this confirmation of specific biomarkers present in menstrual blood will be vital to constructing trials described elsewhere herein, and to define statistically significant elevations of those specific biomarkers found in menstrual blood throughout the duration of the period. The quantitative assessment of these marked elevations will take precedence during the production of subsequent clinical trials described elsewhere herein using identified biomarkers in menstrual blood for early detection of ovarian cancer. CA-125 biomarker elevations will be used as a benchmark against any biomarkers validated in this study, along with subsequent correlation and justification of additional biomarker elevations in patients with early stage ovarian cancer. Additional trials will be conducted that will be inclusive of the remaining number of biomarkers found in menstrual blood, similar to the trials described above as they pertain to malignancies outside the reproductive system and/or general well-being. This panel may be implemented into a personal point-of-care device, to be used monthly by women interested in monitoring their gynecologic health, general health and/or well-being.

The timing of diagnosis of ovarian cancer plays a crucial role in increasing the chances of survival. However, due to the latent nature of the symptoms that accompany ovarian cancer, diagnosis is often at later stages when the cancer has metastasized distant to the ovary and the chance of survival is 17%. The diagnostic protocols currently in use require appointments, blood draws, biopsies and other painful and inconvenient procedures that contribute to delayed diagnosis and high mortality rates. An objective of this Example is to validate a panel of novel biomarkers found in menstrual blood that can be integrated into an “at home” proteomic point-of-care device, to screen for ovarian cancer. Other studies will be conducted that will be inclusive of other proteomic constituents of menstrual blood as they relate to cellular processes associated to other diseases and/or malignancies outside of the reproductive system and/or general well-being. Having a tool whose function is to provide insight into the unknown proteomic changes that occurs prior to the onset of malignancies and/or diseases can provide many benefits to women. By using this device every month, the collection of longitudinal data of biomarkers will give both women and physicians a more accurate diagnostic impression of a woman's gynecologic health, health and well-being using menstrual blood.

Despite many advances in the field of screening diagnostic methodologies for cancer, only 15% of all ovarian cancers are found at a nearly stage when the 5-year relative survival rate is 92%. Unfortunately, almost 70% of women with the common epithelial ovarian cancer are not diagnosed until the disease is advanced in stage when the relative 5-year survival rate is 17%. Predictive and preemptive diagnostics capable of detecting cancer at an early stage would likely improve long-term survivability rates. Specifically for ovarian cancer, the most common cause of mortality as a result of late stage ovarian cancer diagnosis is disseminated carcinomas. Despite decades of research, no diagnostic methodology or screening protocol can produce consistent and accurate diagnosis at an early stage when the chances of survival are high. There are proteomic-screening tests that are capable of detecting ovarian cancer, but there has been no progress in the development of diagnostic screening tool capable of early stage diagnosis as a result of numerous limitations. Important limitations associated with mortality and morbidity stem from the ambiguity in the overexpression of the CA-125 protein during different stages of ovarian cancer, and variances in the expression of this protein amongst individuals during different phases of menstrual cycle. Additional limitations include the latent nature of symptoms associated with ovarian cancer and other malignancies and/or diseases outside of the reproductive system; and inconvenient and painful blood draws and lab procedures that serve no role in increasing patient accountability for their own health.

There is a population of biomarkers within menstrual blood that will overcome these hurdles. For example, activin A and follistatin have an active role in endometrial function and the quantitative assessment of these proteins in menstrual serum illustrates the potential for the use of Activin A and Follistatin as tumor markers for ovarian cancer. There may be a reduction in false-positives while screening for ovarian cancer when Follistatin was combined with CA-125. Therefore, an object of this Example is to provide an at-home diagnostic tool that primarily tests for protein biomarkers using menstrual blood, which can be collected each month, creating a log of longitudinal data that offers more accurate and personalized diagnostics to screen for ovarian cancer. For instance, the following hypotheses are investigated:

Biomarkers found in menstrual fluid are amore accurate medium than peripheral blood to test for neoplastic gynecologic pathologies and other diseases of the female reproductive system due to menstrual fluid's intimate relationship with the female reproductive organs.

Activin A and Follistatin are examples of many biomarkers found in menstrual blood that are statistically superior to biomarkers found in peripheral blood to screen for ovarian cancer and other diseases of the reproductive tract.

Biomarkers found in menstrual blood are also indicative of diseases or general well-being outside of the female reproductive system. Illustrative specific aims include: (1) showing that menstrual fluid can be used to screen for ovarian cancer using statistical and quantitative assessments of biomarkers. An exemplary milestone is elevation of biomarkers associated to ovarian cancer found in menstrual blood is correlated to elevations of biomarkers in peripheral blood; (2) determining the degree of correlation between Activin A and Follistatin in menstrual blood in relation to ovarian cancer. An exemplary milestone is Activin A and Follistatin are elevated in menstrual blood in patients with ovarian cancer.

The use of a diagnostic screening tool that uses menstrual blood to test for biomarkers associated with ovarian cancer should greatly improve patient-accountability, patient quality of life, prognosis of the disease, and reduce the economic burden that accompanies cancer treatment. The device will benefit those by offering personalized longitudinal data collection of biomarkers using menstrual blood to screen for ovarian cancer from a point-of-care device that can be used from the privacy of the patient's home. This shift of more personalized care through portable, modern and private screening diagnostic tool using biosensors and the natural process of menstruation will likely result in more patient accountability and a decrease in mortality as a result of late diagnostics.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. A method for evaluating one or more disease- or health-related biomarkers in a female subject, comprising: (a) obtaining a first sample of the female subject's menstrual fluid on a collection device; (b) measuring the presence, absence, or level of the one or more disease- or health-related biomarkers in the sample; and (c) repeating the sampling of subsequent menstrual fluid samples and measuring the presence, absence, or level of the one or more disease- or health-related biomarkers in the subsequent sample and wherein the method comprises extracting biomarker proteins from the menstrual fluid and examining concentrations of the extracted biomarker proteins to determine the presence, absence, or level of the one or more disease or health related biomarkers.

2. The method of claim 1 wherein the menstrual blood sample is evaluated for the presence, absence or level of alpha and beta glycations of hemoglobin to evaluate the susceptibility of the patient to diabetes or pre-diabetes.

3. A method for evaluating one or more disease- or health-related biomarker proteins in a female subject comprising: (a) obtaining a first sample of the female subject's menstrual fluid on a collection device; (b) measuring the presence. absence, or level of the one or more disease- or health-related biomarker proteins in the sample; and (c) repeating the sampling of subsequent menstrual fluid samples and measuring the presence, absence, or level of the one or more disease- or health-related biomarker proteins in the subsequent sample and wherein the method comprises extracting the biomarker proteins from the menstrual fluid and examining concentrations of the extracted biomarker proteins in the menstrual fluid and examining the concentration of extracted proteins to inform the female subject to make health decisions based on the concentrations of extracted biomarker proteins from the menstrual fluid.

4. The method of claim 3, wherein the female subject obtains multiple samples of menstrual fluid and proteins from the menstrual fluid samples are extracted and the concentration of extracted proteins are compared to each other from multiple samples to observe the increase or decrease in the relative concentration of the alpha and beta glycation of hemoglobin.

5. The method of claim 1 wherein the sample is obtained on a tampon, pad or menstrual cup.

6. The method of claim 5, wherein the sample is obtained about once every month, or about once every other month, or about once every 3 months, or about once every 6 months, or about once every 9 months, or about once every year and proteins are extracted therefrom and the concentration of the extracted proteins are compared to concentrations of extracted proteins from prior samples to determine the increase or decrease in the relative concentrations of the selected extracted biomarker proteins.

7. The method of claim 1, wherein the female subject's menstrual fluid is obtained using a device comprising a disposable cartridge, configured to be insertable into a wireless enabled device to extract protein biomarkers of interest from the sample of menstrual fluid.

8. The method of claim 1 wherein the device is a home instrument wherein proteins of interest are extracted from the menstrual fluid and the concentration of the extracted proteins are detected by the home instrument.

9. The method of claim 1 wherein the biomarker is glycodelin and the method further comprises extracting biomarker proteins from the menstrual fluid and examining the concentrations of the extracted protein for glycodelin.

10. The method of claim 1 wherein the biomarker is fibrinogen and the method further comprises extracting biomarker proteins from the menstrual fluid and examining the concentrations of the extracted protein for fibrinogen.

11. The method of claim 1 wherein the biomarker is peroxiredoxin-2 and the method further comprises extracting biomarker proteins from the menstrual fluid and examining the concentrations of the extracted protein for peroxiredoxin-2.

12. The method of claim 1 wherein the biomarker is Lon protease homolog 2, peroxisomal and the method further comprises extracting biomarker proteins from the menstrual fluid and examining the concentrations of the extracted protein for Lon protease homolog 2, peroxisomal.

13. A method for evaluating one or more disease- or health-related biomarkers in a female subject, comprising: (a) obtaining a first sample of the female subject's menstrual fluid; (b) measuring the presence, absence, or level of the one or more disease- or health-related protein biomarkers in the sample; and wherein the method further comprises extracting proteins from subsequent samples of the menstrual fluid in a female subject and examining the concentrations of the extracted protein biomarkers wherein the female subject's biomarker information provides baseline, short-term or long-term trend health information related to the detection of ovarian cancer.

14. The method of claim 1, wherein the baseline, short-term or long-term trend health information is used to compare to a biomarker measurement at a single point of time using dried blood spot analysis protein analysis.

15. The method of claim 1 wherein the biomarker is phosphatidylinositol 4-phosphate 5-kinase type-1 alpha (PIP5K1a) and the method further comprises extracting biomarker proteins from the menstrual fluid and examining the concentrations of the extracted protein for phosphatidylinositol 4-phosphate 5-kinase type-1 alpha (PIP5K1a).

16. The method of claim 1 wherein the biomarker is serotransferrin and the method further comprises extracting biomarker proteins from the menstrual fluid and examining the concentrations of the extracted protein for serotransferrin.

17. The method of claim 1 wherein the biomarker is Protein TANC1 and the method further comprises extracting biomarker proteins from the menstrual fluid and examining the concentrations of the extracted protein for Protein TANC1.

18. The method of claim 1 wherein the biomarker is Teashirt homolog 2 and the method further comprises extracting biomarker proteins from the menstrual fluid and examining the concentrations of the extracted protein for Teashirt homolog 2.

19. The method of claim 1 wherein the biomarker is estrogen and the method further comprises extracting biomarker proteins from the menstrual fluid and examining the concentrations of the extracted protein for estrogen.

20. The method of claim 1, wherein the method further comprises providing a database for the storage and analysis of the subject's biomarker information.
